SEQUENCE LISTING (26 sequences)

SEQ ID NO: 1 - DNA, 21 bp, Artificial Sequence
  synthetic construct
ggctaaacaggtactgctggg

SEQ ID NO: 2 - DNA, 23 bp, Artificial Sequence
  synthetic construct
aggaaagccaggtacttcaactt

SEQ ID NO: 3 - DNA, 20 bp, Artificial Sequence
  synthetic construct
gcagaagaggatcaccctgg

SEQ ID NO: 4 - DNA, 21 bp, Artificial Sequence
  synthetic construct
ttggctgaataccttcccaaa

SEQ ID NO: 5 - DNA, 15 bp, Artificial Sequence
  synthetic construct
ccagtgggcaggcgg

SEQ ID NO: 6 - DNA, 21 bp, Artificial Sequence
  synthetic construct
aggaactccttctccagctct

SEQ ID NO: 7 - DNA, 20 bp, Artificial Sequence
  synthetic construct
ggcctgtacccctcatcaag

SEQ ID NO: 8 - DNA, 20 bp, Artificial Sequence
  synthetic construct
tccggaaaaagtgggtctcg

SEQ ID NO: 9 - DNA, 20 bp, Artificial Sequence
  synthetic construct
aggccatcaagcagatcact

SEQ ID NO: 10 - DNA, 20 bp, Artificial Sequence
  synthetic construct
ttccccgcacactaggtaga

SEQ ID NO: 11 - DNA, 20 bp, Artificial Sequence
  synthetic construct
ccggtagaaaggatgacgcc

SEQ ID NO: 12 - DNA, 20 bp, Artificial Sequence
  synthetic construct
ggtcacttcgtcttccgagg

SEQ ID NO: 13 - DNA, 20 bp, Artificial Sequence
  synthetic construct
actccgaggagatgaacgga

SEQ ID NO: 14 - DNA, 20 bp, Artificial Sequence
  synthetic construct
tggctgctctggttctcaag

SEQ ID NO: 15 - DNA, 20 bp, Artificial Sequence
  synthetic construct
cagccacaagttcatgggga

SEQ ID NO: 16 - DNA, 20 bp, Artificial Sequence
  synthetic construct
atctctccccgagagtggac

SEQ ID NO: 17 - DNA, 27 bp, Artificial Sequence
  synthetic construct
ggtcagcagcatcgtggtcaacataac

SEQ ID NO: 18 - DNA, 26 bp, Artificial Sequence
  synthetic construct
tggagcaggacaggttcagtctttca

SEQ ID NO: 19 - DNA, 21 bp, Artificial Sequence
  synthetic construct
tcccttccttcagtcatgtgt

SEQ ID NO: 20 - DNA, 22 bp, Artificial Sequence
  synthetic construct
aagaaggaaacagaatctgcaa

SEQ ID NO: 21 - DNA, 28 bp, Artificial Sequence
  synthetic construct
agccatgtggtctctctggttgtgtatg

SEQ ID NO: 22 - DNA, 26 bp, Artificial Sequence
  synthetic construct
gtttgagtggtgccgtactggtagga

SEQ ID NO: 23 - DNA, 20 bp, Artificial Sequence
  synthetic construct
catcttcccaggaggaacag

SEQ ID NO: 24 - DNA, 20 bp, Artificial Sequence
  synthetic construct
agagctccactcacgctcag

SEQ ID NO: 25 - DNA, 20 bp, Artificial Sequence
  synthetic construct
ctccaccatgccaagtggtc

SEQ ID NO: 26 - DNA, 20 bp, Artificial Sequence
  synthetic construct
gcagtagctgcgctgataga

(19) United States
(12) Patent Application Publication
(10) Pub. No.: US 2024/0093154 A1
(43) Pub. Date: Mar. 21, 2024

(54) GENERATION OF FUNCTIONAL HUMAN IPSC-DERIVED PANCREATIC ISLETS IN CO-CULTURE WITH ISOGENIC IPSC-DERIVED VASCULAR ENDOTHELIAL CELLS

(71) Applicant: CEDARS-SINAI MEDICAL CENTER, Los Angeles, CA (US)

(72) Inventors: Dhruv Sareen, Porter Ranch, CA (US); Roberta de Souza Santos, Los Angeles, CA (US)

(73) Assignee: CEDARS-SINAI MEDICAL CENTER, Los Angeles, CA (US)

(21) Appl. No.: 18/272,969

(22) PCT Filed: Jan. 31, 2022
(86) PCT No.: PCT/US2022/014630

§ 371 Date: Jul. 18, 2023

Related U.S. Application Data
(60) Provisional application No. 63/144,155, filed on Feb. 1, 2021.

(51) Int. Cl. C12N 5/071; A61K 35/39; A61P 3/10; C12N 5/00

(52) U.S. Cl. CPC C12N 5/0676; A61K 35/39; A61P 3/10; C12N 5/0018

(57) ABSTRACT

Diabetes is a clinical condition that affects millions of people worldwide, and is treated by insulin replacement therapies. New strategies to create scalable and compatible pancreatic islets containing insulin-producing beta cells are necessary as an alternative to limited supply of cadaveric islets or multiple exogenous insulin applications. Improvements are still necessary since many immature polyhormonal cells remain, and cannot attain a monohormonal state. During human development, pancreas co-develops with endothelium and shares signals, allowing for better maturation of beta cells, and this is not included in the current differentiation protocols. The organchip microfluidic devices allows dynamic co-culture of different cells, thus resembling in vivo physiology. Here the Inventors establish organ-chip models co-culturing human iPSC-derived pancreatic precursors with iPSC-derived endothelial cells to obtain more functional and monohormonal iPSC-derived beta cells.

### CROSS-REFERENCE TO RELATED APPLICATIONS

This application includes a claim of priority under 35 U.S.C. § 119(e) to U.S. provisional patent application No. 63/144,155, filed Feb. 1, 2021, the entirety of which is hereby incorporated by reference.

### STATEMENT REGARDING FEDERALLY SPONSORED RESEARCH OR DEVELOPMENT

This invention was made with government support under Grant No. DK063491 awarded by National Institutes of Health. The government has certain rights in the invention.

### FIELD OF THE INVENTION

The present invention relates to the field of culturing cells, and in particular, culturing islet cells together with other cell types.

### BACKGROUND

Diabetes affects millions of people worldwide and is mainly characterized by hyperglycemia due to dysfunctions of pancreatic islets that produce little to no amounts of insulin that are insufficient to the bodily demand of glucose. Most of the patients with diabetes are reliant on multiple exogenous insulin injections that can cause adverse effects, and some of them are recipients of cadaveric islet transplantation, which is a scarce source and require long-term immunosuppression. Thus, novel strategies to create scalable and compatible pancreatic islets containing insulin-producing β-cells are in great need.

### SUMMARY OF THE INVENTION

The following embodiments and aspects thereof are described and illustrated in conjunction with compositions and methods which are meant to be exemplary and illustrative, not limiting in scope.

Various embodiments of the invention provide for a method of generating functional induced pluripotent stem cell (iPSC) derived pancreatic islets (iIslets), comprising: co-culturing a quantity of iPSC derived vascular endothelial cells (iECs) and a quantity of iPSC derived islet progenitors for about 10-18 days to generate the functional iIslets comprising β-cells.

In various embodiments, the co-culturing can comprise: plating a quantity of iPSC derived vascular endothelial cells (iECs) on MATRIGEL-coated plates and culturing in Phase IV EC media supplemented with Y27632; plating a quantity of iPSC derived pancreatic islets (iIslets) on top of the quantity of iECs and either culturing in media comprising about ½ Phase IV iEC media and about ½ Phase VI islet media supplemented with Y-27632 for about 12-16 days, or culturing in Phase VI islet media (islet only condition) for about 12-16 days, to generate the functional iIslets comprising β-cells.

In various embodiments, the co-culturing can comprise: plating a quantity of iPSC derived vascular endothelial cells (iECs) on MATRIGEL-coated plates and culturing in Phase IV EC media supplemented with Y27632; plating a quantity of iPSC derived pancreatic islets (iIslets) on top of the quantity of iECs and either culturing in media comprising about ½ Phase IV iEC media and about ½ Phase VI islet media supplemented with Y-27632 for about 14 days, or culturing in Phase VI islet media (islet only condition) for about 14 days, to generate the functional iIslets comprising β-cells.

In various embodiments, the method can further comprise first generating the iECs by: plating a quantity of induced pluripotent stem cells (iPSCs) onto MATRIGEL; culturing the iPSC in MATRIGEL for about 2-4 days; culturing in the presence of CHIR99021 for about 1-3 days to generate mesoderm; culturing the mesoderm in the presence of BMP4, FGF2, and VEGF for about 1-3 days to generate vascular progenitors; culturing the vascular progenitors in the presence of VEGF and Y-27632 for about 3-8 days to generate endothelial cell (EC) progenitors.

In various embodiments, the method can further comprise first generating the iECs by: plating a quantity of induced pluripotent stem cells (iPSCs) onto MATRIGEL; culturing the iPSC in MATRIGEL for about 3 days; culturing in the presence of CHIR99021 for about 2 days to generate mesoderm; culturing the mesoderm in the presence of BMP4, FGF2, and VEGF for about 2 days to generate vascular progenitors; culturing the vascular progenitors in the presence of VEGF and Y-27632 for about 4-7 days to generate endothelial cell (EC) progenitors.

In various embodiments, the method can further comprise first generating the quantity of islet progenitors by: culturing a quantity of induced pluripotent stem cells (iPSCs) in the presence of Activin-A, CHIR99021 and Y-27632 for about 1-2 days; culturing in the presence of Activin-A and FGF2 for about 1-3 days; culturing in the presence of FGF10, CHIR99021 and Noggin for about 1-3 days, to generate posterior foregut cells; culturing the posterior foregut cells in the presence of FGF10, Noggin, RA and SANT1 for about 3-5 days to generate pancreatic progenitors; culturing the pancreatic progenitors in the presence of Noggin, EGF and Nicotinamide for about 3-5 days to generate pancreatic endocrine progenitors; culturing the pancreatic endocrine progenitors in the presence of Noggin, T3 and Alk5i II for about 6-8 days to generate islet progenitors.

In various embodiments, the method can further comprise first generating the quantity of islet progenitors by: culturing a quantity of induced pluripotent stem cells (iPSCs) in the presence of Activin-A, CHIR99021 and Y-27632 for about 1 day; culturing in the presence of Activin-A and FGF2 for about 2 days; culturing in the presence of FGF10, CHIR99021 and Noggin for about 2 days, to generate posterior foregut cells; culturing the posterior foregut cells in the presence of FGF10, Noggin, RA and SANT1 for about 4 days to generate pancreatic progenitors; culturing the pancreatic progenitors in the presence of Noggin, EGF and Nicotinamide for about 4 days to generate pancreatic endocrine progenitors; and culturing the pancreatic endocrine progenitors in the presence of Noggin, T3 and Alk5i II for about 7 days to generate islet progenitors.

In various embodiments, the pancreatic progenitors can express PDX1+ and SOX9+.

In various embodiments, the pancreatic endocrine progenitors can be PDX1+ and NKX6.1+.

In various embodiments, the iIslets can express C-peptide, glucagon and NKX6.1+.

In various embodiments, the expression of INS, UCN3, NGN3 and CHGA can be upregulated in the β-cell that are produced in the islets only condition, as compared to β-cell that are produced without co-culturing with vascular endothelial cells or as compared to β-cell that were produced in a culture without the islets only condition.

In various embodiments, the β-cell can increase insulin secretion when challenged with a high glucose concentration as compared to a basal glucose concentration.

In various embodiments, the iPSC derived vascular endothelial cells (iECs) and iPSC derived islet progenitors can be isogenic.

In various embodiments, the iPSCs used to derive vascular endothelial cells (iECs) and iPSC used to derive islet progenitors can be from the same iPSC cell line or from the same donor.

In various embodiments, the iIslets can be human iIslets.

Various embodiments of the invention provide for induced pluripotent stem cell (iPSC) derived pancreatic islets (iIslets) produced by any one of the methods as described herein. In various embodiments, the induced pluripotent stem cell (iPSC) derived pancreatic islets (iIslets) can express C-peptide, glucagon and NKX6.1+. In various embodiments, the iIslets can increase insulin secretion when challenged with a high glucose concentration as compared to a basal glucose concentration.

Various embodiments of the invention provide for a method of ameliorating or treating a metabolic disease, metabolic disorder or metabolic condition in a subject in need thereof, comprising: administering iIslets of the present invention to the subject in need thereof to ameliorate or treat the metabolic disease, metabolic disorder or metabolic condition. In various embodiments, the metabolic disease, metabolic disorder or metabolic condition can be diabetes or insulin resistance.

Various embodiments of the invention provide for a method, comprising: culturing a quantity of induced pluripotent stem cells (iPSCs) in the presence of Activin-A, CHIR99021 and Y-27632 for about 1 day; followed by culturing in the presence of Activin-A and FGF2 for about 2 days; and followed by culturing in the presence of FGF10, CHIR99021 and Noggin for about 2 days, to generate posterior foregut cells.

In various embodiments, the method can further comprise culturing the posterior foregut cells in the presence of FGF10, Noggin, RA and SANT1 for about 4 days to generate pancreatic progenitors.

In various embodiments, the pancreatic progenitors can express PDX1⁺ and SOX9⁺.

In various embodiments, the method can further comprise culturing the pancreatic progenitors in the presence of Noggin, EGF and Nicotinamide for about 4 days to generate pancreatic endocrine progenitors.

In various embodiments, the pancreatic endocrine progenitors can be PDX1⁺ and NKX6.1⁺.

In various embodiments, the method can further comprise comprising culturing the pancreatic endocrine progenitors in the presence of Noggin, T3 and Alk5i II for about 7 days to generate islet progenitors.

In various embodiments, the method can further comprise culturing the generated islet progenitors in the presence of T3, Alk5i II, R428, and N-acetylcysteine (NAC) for about 14 days to generate mature islets.

In various embodiments, the mature islets can express C-peptide, glucagon and NKX6.1⁺.

In various embodiments, the method can further comprise culturing the posterior foregut in the presence of FGF10, Noggin, RA and SANT1 for about 4 days to generate pancreatic progenitors; followed by culturing the pancreatic progenitors in the presence of Noggin, EGF and Nicotinamide for about 4 days to generate pancreatic endocrine progenitors; followed by culturing the pancreatic endocrine progenitors in the presence of Noggin, T3 and Alk5i II for about 7 days to generate islet progenitors; and followed by culturing the generated islet progenitors in the presence of T3, Alk5i II, R428, and N-acetylcysteine (NAC) for about 14 days to generate mature islets.

Various embodiments of the present invention provide for a quantity of mature islets made by any one of the methods of the present invention described herein.

Various embodiments of the present invention provide for a method, comprising: plating a quantity of induced pluripotent stem cells (iPSCs) onto MATRIGEL; culturing for about iPSC in MATRIGEL for about 3 days; and followed by culturing in the presence of CHIR99021 to generate mesoderm.

In various embodiments, the method can further comprise the mesoderm in the presence of BMP4, FGF2, and VEGF for about 2 days to generate vascular progenitors.

In various embodiments, the method can further comprise culturing the vascular progenitors in the presence of VEGF and Y-27632 for about 7 days to generate endothelial cell (EC) progenitors.

In various embodiments, the method can further comprise culturing the EC progenitors with VEGF for about 10 days to generate mature EC. In various embodiments, the mature EC express CD31⁺, CD144⁺, VEGF-A⁺, VEGFR2⁺, and Ac-LDL.

In various embodiments, the method can further comprise culturing the mesoderm in the presence of BMP4, FGF2, and VEGF for about 2 days to generate vascular progenitors; followed by culturing the vascular progenitors in the presence of VEGF and Y-27632 for about 7 days to generate endothelial cell (EC) progenitors; and followed by culturing the EC progenitors with VEGF for about 10 days to generate mature EC.

Various embodiments of the invention provide for a quantity of mature EC made by any one of the methods of the present invention described herein.

Various embodiments of the invention provide an assembly, comprising a quantity of mature islets and a quantity of mature EC, wherein the mature islets and the mature EC are isogenic.

In various embodiments, the quantity of the mature islets can comprise mature islets of the present invention as described herein, and the quantity of the mature ECs can comprise mature ECs of the present invention as described herein.

In various embodiments, the mature islets, the mature EC, or both, can be deposited on a scaffold. In various embodiments, the mature islets, mature EC or both, can be deposited on the scaffold using a bioink. In various embodiments, the bioink can comprise fibrin or alginate.

Also described herein is a method, including providing a quantity of induced pluripotent stem cells (iPSCs), culturing in the presence of Activin-A, CHIR99021 and Y-27632 for about 1 day, further culturing in the presence of Activin-A and FGF2 for about 2 days, additionally culturing in the presence of FGF10, CHIR99021 and Noggin for about 2 days, to generate posterior foregut.

In other embodiments, the method includes culturing the posterior foregut in the presence of FGF10, Noggin, RA and SANT1 for about 4 days to generate pancreatic progenitors. In other embodiments, the pancreatic progenitors express PDX1⁺ and SOX9⁺. In other embodiments, the method includes culturing the pancreatic progenitors in the presence of Noggin, EGF and Nicotinamide for about 4 days to generate pancreatic endocrine progenitors. In other embodiments, the pancreatic endocrine progenitors are PDX1⁺ and NKX6.1⁺. In other embodiments, the method includes culturing the pancreatic endocrine progenitors in the presence of Noggin, T3 and Alk5i II for about 7 days to generate islet progenitors. In other embodiments, the method includes culturing the generate islet progenitors in the presence of T3, Alk5i II, R428, and N-acetylcysteine (NAC) for about 14 days to generate mature islets. In other embodiments, the mature islets express C-peptide, glucagon and NKX6.1⁺.

Also described herein is a quantity of mature islets made by the method, including providing a quantity of induced pluripotent stem cells (iPSCs), culturing in the presence of Activin-A, CHIR99021 and Y-27632 for about 1 day, further culturing in the presence of Activin-A and FGF2 for about 2 days, additionally culturing in the presence of FGF10, CHIR99021 and Noggin for about 2 days, to generate posterior foregut. In other embodiments, the method includes culturing the posterior foregut in the presence of FGF10, Noggin, RA and SANT1 for about 4 days to generate pancreatic progenitors. In other embodiments, the pancreatic progenitors express PDX1⁺ and SOX9⁺. In other embodiments, the method includes culturing the pancreatic progenitors in the presence of Noggin, EGF and Nicotinamide for about 4 days to generate pancreatic endocrine progenitors. In other embodiments, the pancreatic endocrine progenitors are PDX1⁺ and NKX6.1⁺. In other embodiments, the method includes culturing the pancreatic endocrine progenitors in the presence of Noggin, T3 and Alk5i II for about 7 days to generate islet progenitors. In other embodiments, the method includes culturing the generate islet progenitors in the presence of T3, Alk5i II, R428, and N-acetylcysteine (NAC) for about 14 days to generate mature islets. In other embodiments, the mature islets express C-peptide, glucagon and NKX6.1⁺.

Also described herein is a method, including providing a quantity of induced pluripotent stem cells (iPSCs), plating the iPSCs onto MATRIGEL, culturing for about iPSC in MATRIGEL for about 3 days, further culturing in the presence of CHIR99021 to generate mesoderm. In other embodiments, the method includes culturing the mesoderm in the presence of BMP4, FGF2, and VEGF for about 2 days to generate vascular progenitors. In other embodiments, the method includes culturing the vascular progenitors in the presence of VEGF and Y-27632 for about 7 days to generate endothelial cell (EC) progenitors. In other embodiments, the method includes culturing the EC progenitors with VEGF for about 10 days to generate mature EC. In other embodiments, the method includes culturing vascular progenitors in the presence of EGM-MV2 and VEGF for about 4-6 days to generate endothelial progenitor cells, and culturing endothelial progenitor cells in the presence of EGM-MV2 and VEGF to generate endothelial cells. In other embodiments, the mature EC express CD31⁺, CD144⁺, VEGF-A⁺, VEGFR2⁺, and Ac-LDL.

Also described herein is a quantity of mature EC made by the method, including providing a quantity of induced pluripotent stem cells (iPSCs), plating the iPSCs onto MATRIGEL, culturing for about iPSC in MATRIGEL for about 3 days, further culturing in the presence of CHIR99021 to generate mesoderm. In other embodiments, the method includes culturing the mesoderm in the presence of BMP4, FGF2, and VEGF for about 2 days to generate vascular progenitors. In other embodiments, the method includes culturing the vascular progenitors in the presence of VEGF and Y-27632 for about 7 days to generate endothelial cell (EC) progenitors. In other embodiments, the method includes culturing the EC progenitors with VEGF for about 10 days to generate mature EC. In other embodiments, the method includes culturing vascular progenitors in the presence of EGM-MV2 and VEGF for about 4-6 days to generate endothelial progenitor cells, and culturing endothelial progenitor cells in the presence of EGM-MV2 and VEGF to generate endothelial cells. In other embodiments, the mature EC express CD31⁺, CD144⁺, VEGF-A⁺, VEGFR2⁺, and Ac-LDL.

Also described herein is an assembly, including a quantity of mature islets and a quantity of mature EC. In other embodiments, the mature islets are made by the method, including providing a quantity of induced pluripotent stem cells (iPSCs), culturing in the presence of Activin-A, CHIR99021 and Y-27632 for about 1 day, further culturing in the presence of Activin-A and FGF2 for about 2 days, additionally culturing in the presence of FGF10, CHIR99021 and Noggin for about 2 days, to generate posterior foregut. In other embodiments, the method includes culturing the posterior foregut in the presence of FGF10, Noggin, RA and SANT1 for about 4 days to generate pancreatic progenitors. In other embodiments, the pancreatic progenitors express PDX1⁺ and SOX9⁺. In other embodiments, the method includes culturing the pancreatic progenitors in the presence of Noggin, EGF and Nicotinamide for about 4 days to generate pancreatic endocrine progenitors. In other embodiments, the pancreatic endocrine progenitors are PDX1⁺ and NKX6.1⁺. In other embodiments, the method includes culturing the pancreatic endocrine progenitors in the presence of Noggin, T3 and Alk5i II for about 7 days to generate islet progenitors. In other embodiments, the method includes culturing the generate islet progenitors in the presence of T3, Alk5i II, R428, and N-lacetylcysteine (NAC) for about 14 days to generate mature islets. In other embodiments, the mature islets express C-peptide, glucagon and NKX6.1⁺. In other embodiments, the mature EC are made by the method, including providing a quantity of induced pluripotent stem cells (iPSCs), plating the iPSCs onto MATRIGEL, culturing for about iPSC in MATRIGEL for about 3 days, further culturing in the presence of CHIR99021 to generate mesoderm. In other embodiments, the method includes culturing the mesoderm in the presence of BMP4, FGF2, and VEGF for about 2 days to generate vascular progenitors. In other embodiments, the method includes culturing the vascular progenitors in the presence of VEGF and Y-27632 for about 7 days to generate endothelial cell (EC) progenitors. In other embodiments, the method includes culturing the EC progenitors with VEGF for about 10 days to generate mature EC. In other embodiments, the method includes culturing vascular progenitors in the presence of EGM-MV2 and VEGF for about 4-6 days to generate endothelial progenitor cells, and culturing endothelial progenitor cells in the presence of EGM-MV2 and VEGF to generate endothelial cells. In other embodiments, the mature EC express CD31⁺, CD144⁺, VEGF-A⁺, VEGFR2⁺, and Ac-LDL. In other embodiments, the mature islets and mature EC are isogenic.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

FIG. 1 depicts protein expression of endocrine pancreatic progenitor markers.

FIG. 2 depicts mRNA expression of pluripotency (Oct4) and endocrine pancreatic (Pdx1, Nkx6.1, C-peptide) markers throughout development.

FIG. 3 depicts protein expression of endothelial cell markers.

FIG. 4 depicts mRNA expression of endothelial cell markers.

FIGS. 5A-5B depict results from co-culture systems. (5A) Representation of co-culture systems (5B) GSIS results using direct co-culture of iECs with iIslets.

FIG. 6 depicts mRNA expression of β-cell markers.

FIGS. 7A-7D depict a perfusion system to be adapted to a GSIS dynamic system. (A) Perfusion system flowing media to the cells. (B) iECs cultured in planar to be transferred to 3D-printed vessel constructs. (C), (D) iECs remained attached to vessel constructs casts for at least 2 weeks.

FIGS. 8A-8C depict successful generation of iPSC-derived Pancreatic Endocrine Progenitors. 8A. Schematic protocol of iPSC differentiation into to pancreatic endocrine progenitor (PEP) phase with small molecules in detail. 8B. Immunofluorescence images showing the expression of markers for each stage: OCT4/SSEA4 for iPSC; FOXA2/SOX17 for DE; PDX1/SOX9 for PP; and PDX1/NKX6.1 for PEP. For this experiment, 3 different cell lines were used, as indicated. Cropped images (insets) corresponds to 1/7 of original image. 8C. mRNA expression of different markers for each stage, as indicated. This experiment was performed with 07iCTR-n07 cell line. Data are shown as mean±SEM (n=3/experiment). * P<0.05 vs iPSC. DE: definitive endoderm. PFG: posterior foregut. PP: pancreatic progenitor. PEP: pancreatic endocrine progenitor. Nicotin.: nicotinamide.

FIGS. 9A-9D depict generation of iPSC-derived Islet Progenitors. 9A. Schematic representation of the main signaling pathways that were tested out to optimize the generation of islet progenitors (IPs) from pancreatic endocrine progenitors (PEPs). 9B. The combination of small molecules that were tested to modulate the main pathways were as follows: i) Alk5i and T3; ii) Alk5i, T3 and Noggin; iii) Alk5i, T3 and XXI; and iv) Alk5i, T3, Noggin and XXI. Immunofluorescence images showing the expression of markers of pancreatic islets: C-PEPTIDE (C-PEP) and NKX6.1 for β-cells; Glucagon (GCG) for α cells and Somatostatin (SST) for δ cells. Cropped images (insets) corresponds to 1/7 of original image. 9C. Number of cells per well for each of the conditions in B. 9D. Percentage of expression of each of the markers in B and combinations of them. These experiments were performed with 07iCTR-n07 cell line. Data are shown as mean±SEM (n=3/experiment). * P<0.05 vs Alk5i, T3; \hat{ } P<0.05 vs Alk5i, T3, Noggin; ^{#}P<0.05 vs Alk5i, T3, XXI. PEP: pancreatic endocrine progenitor. IP: islet progenitor. Nog: Noggin.

FIGS. 10A-10D depict optimization of the maturation process of iPSC-derived mature islets (MIs). 10A. Schematic representation of main signaling pathways that were tested out to optimize the maturation of islet progenitors (IPs) into mature islets (MIs). 10B. The combination of small molecules that were tested out to modulate the main pathways were as follows: i) T3; ii) T3 and Alk5i; iii) T3, Alk5i and NAC; and iv) T3, Alk5i, NAC and R428. Immunofluorescence images showing the expression of markers of pancreatic islets: C-PEPTIDE (C-PEP) and Nkx6.1 for β-cells; Glucagon (GCG) for α cells and Somatostatin (SST) for δ cells. Cropped images (insets) corresponds to 1/7 of original image. 10C. Number of cells per well for each of the conditions in B. 10D. Percentage of expression of each of the markers in B. These experiments were performed with 07iCTR-n07 cell line. Data are shown as mean±SEM (n=3/experiment). *P<0.05 vs T3; \hat{ }P<0.05 vs T3, Alk5i. PEP: pancreatic endocrine progenitor. NAC: N-acetyl cysteine. R248: AXL inhibitor R428.

FIGS. 11A-11D depict successful generation of iPSC-derived vascular endothelial cells (iECs). 11A. Schematic protocol of iPSC differentiation into vascular endothelial cells (iECs). 11B. Immunofluorescence images showing the expression of endothelial markers at Day 21: VEGFR2/CD31 and CD144/CD31, and 11C. % of expression of DAPI cells. Cells were also assayed for functionality with Dil-Acil-LDL uptake assay (11B-11C). 11D. mRNA expression of different endothelial markers at different days of differentiation. This experiment was performed with 07iCTR-n07 cell line. Data are shown as mean±SEM (n=3/experiment). *p<0.05 vs iPSC.

FIGS. 12A-12D depict direct co-culture of i-mature islets (i-MIs) with iECs leads to enhancement of i-MIs functionality. 12A. Schematic of direct co-culture: i-MIs were co-cultured on top of iECs for 2 weeks fed with Phase VI i-MI media. 12B. Immunofluorescence images showing expression of pancreatic endocrine markers C-PEPTIDE and NKX.6.1 in i-MIs cultured alone or in co-cultured with iECs. The fold change expression of C-PEPTIDE/NKX6.1 doubled when i-MIs were in co-culture. 12C. Cells were assayed for functionality through glucose stimulated insulin secretion (GSIS). Fold change of insulin secretion is shown in this graph comparing i-MIs alone vs i-MIs co-cultured with iECs, challenged to 2.8 mM glucose and 20 mM glucose. 12D. mRNA expression of different pancreatic endocrine markers after 2 weeks of co-culture. This experiment was performed with 07iCTR-n07 cell line. Data are shown as mean±SEM (n=3/experiment). *p<0.05 vs 2.8 mM glucose within i-MIs only; ^{#}p<0.05 vs 20 mM i-MIs alone for FIG. 5C. *<0.05 vs iPSCs; \hat{ }p<0.05 vs i-MIs alone for FIG. 12D.

FIGS. 13A-13B depict morphology of cells throughout iEC differentiation. 13A. Brightfield images of iPSC colonies at Day 3 after being passaged and plated onto MATRIGEL-coated plates. 13B. Brightfield images of Vascular progenitors (VPs) at the end of Phase II of iEC differentiation. VPs of the periphery lift easily after dissociation and can be re-plated in planar onto MATRIGEL-coated plates to be induced into endothelial cell progenitors (ECs) during Phase III of iEC differentiation.

FIG. 14A-14C depict quantification of protein expression of markers of pancreatic endocrine progenitors (PEP) across different cell lines. Quantification of protein expression of pancreatic endocrine markers PDX1 and NKX6.1 in three cell lines: 07iCTR-n07, EDi028-A and EDi029-A (% of expression of DAPI cells). Data are shown as mean±SEM (n=3/experiment). *p<0.05, **p<0.01, ****p<0.0001.

FIG. 15 depicts gene expression of pluripotency marker OCT4 across iPSC differentiation into pancreatic endocrine progenitors (PEPs). Real time qPCR (RT-qPCR) was performed with 07iCTR-n05, NCI-N87 and MKN-45 human gastric cancer cell lines. Data are shown as mean±SEM (n=3/experiment). * P<0.05 vs iPSC.

FIG. 16 depicts static glucose stimulating insulin secretion (GSIS) on islet progenitors (IPs). Islet progenitors (IPs) were challenged to a static GSIS assay, which consisted in the stimulation of cells with 2.8 mM glucose solution (1 hr), followed by 20 mM glucose solution (1 hr), and at the end, 30 mM KCl solution stimulation (1 hr). This experiment was performed with 07iCTR-n07 cell line. Data are shown as mean±SEM (n=3/experiment). * P<0.05 vs Alk5i, T3 condition; ^{&}P<0.05 vs 2.8 mM glucose.

FIG. 17 depicts static glucose stimulating insulin secretion (GSIS) on mature islets (Ms). Mature islets (Ms) were challenged to a GSIS assay, which consisted in the stimulation of cells with 2.8 mM glucose solution (1 hr), followed by 20 mM glucose solution (1 hr), and at the end, 30 mM KCl solution stimulation (1 hr). This experiment was performed with 07iCTR-n07 cell line. Data are shown as mean±SEM (n=3/experiment). *P<0.05 vs T3 condition. \hat{ }P<0.05 vs T3, Alk5i condition. ^{#}P<0.05 vs T3, Alk5i, NAC condition; ^{&}P<0.05 vs 2.8 mM glucose.

FIG. 18A-18B depict testing out different medium conditions on Phase VI to increase functionality of iIslets. 18A. Schematic representation of the main factors to be modulated in the medium to optimize the generation of functional Mature Islets. 18B. We have tested out the conditions: i) Medium with 8 mM glucose and ITS-X; ii) Medium with 8 mM glucose and no ITS-X; iii) Medium with 20 mM glucose and ITS-X; and iv) Medium with 20 mM glucose and no ITS-X. Immunofluorescence images showing the expression of markers of pancreatic islets: C-PEPTIDE (C-PEP) and NKX6.1 for β-cells, Glucagon (GCG) for α cells and Somatostatin (SST) for δ cells. Cropped images (insets) corresponds to 1/7 of original image. Data are shown as mean±SEM (n=3/experiment). IP: islet progenitor. MI: mature islet. Gluc: glucose. ITS-X: insulin-transferrin-selenium-ethanol amine.

FIG. 19A-19C depict testing out reaggregation of mature islets (Mis) and timing to increase functionality of functional iIslets. 19A. Schematic representation of the main factors to be modulated in order to optimize generation of functional Mature iIslets. 19B. We have tested out the conditions: i) Reaggregation of cells at first day of Phase VI followed by 7 days of cell culture; and ii) Reaggregation of cells at first day of Phase VI followed by 14 days of cell culture. Immunofluorescence images showing the expression of markers of pancreatic islets: C-PEPTIDE (C-PEP) and NKX6.1 for β-cells, and Glucagon (GCG) for α cells. Cropped images (insets) corresponds to 1/7 of original image. 19C. Cells were challenged to static glucose stimulated insulin secretion (GSIS), which consisted in the stimulation of cells with 2.8 mM glucose solution (1 hr), followed by 20 mM glucose solution (1 hr), and at the end, 30 mM KCl solution stimulation (1 hr). These experiments were performed with 07iCTR-n07 cell line. Data are shown as mean±SEM (n=3/experiment). *P<0.05 vs 2.8 mM glucose; \hat{ }P<0.05 vs 20 mM glucose, ^{&}P<0.05 vs Reag. Day 28. IP: islet progenitor. MI: mature islet. Reag.: reaggregation.

FIG. 20 depicts other cell types after reaggregation might influence lack of functionality of mature islets. Immunofluorescence images showing the expression of markers of pancreatic islets at Day 35 post-reaggregation: C-PEPTIDE (C-PEP) and KX6.1 for β-cells, Glucagon (GCG) for α cells, PDX1/SOX9 for pancreatic progenitors (PPs), PDX1/CK19 for pancreatic ductal cells, and PDX1/CDX2 as a stomach/intestine marker. Cropped images (insets) corresponds to 1/7 of original image. These experiments were performed with 07iCTR-n07 cell line. Data are shown as mean±SEM (n=3/experiment). Reag.: reaggregation.

FIG. 21 depicts iEC's pluripotency.

FIGS. 22A-22B depict generation of protocol to differentiate iPSCs into endothelial cells (22A) schematic of the protocol, (22B) protein expression of main vascular endothelial markers of iECs at Day 21 in 5 different iPSC lines.

FIGS. 23A-23B depict further characterization of key markers of iECs (22A) protein expression of endothelial markers CD31 and CD144 assessed by flow cytometry at Day 11 (22B) gene expression of pluripotent markers OCT4 and SOX2 assess by RT-qPCR in multiple days of iEC differentiation.

FIGS. 24A-24B depict addition of Angiopoietin-1 to enhance expression of VEGFA and CD31 iEC markers by day 11 (24A) protein expression of endothelial markers VEGFA and CD31 at day 11 (24B) percentage of cells expressing iEC markers in different conditions.

FIGS. 25A-25B depict addition of Angiopoietin-1 to enhance expression of CD144 marker by day 11 (25A) protein expression of endothelial markers CD144 at day 11 (25B) percentage of cells expressing iEC markers in different conditions.

FIGS. 26A-26B depict addition of Angiopoietin-1 to enhance expression of VEGFA and CD31 iEC markers by day 21. (26A) protein expression of endothelial markers VEGFA and CD31 at day 21. (26B) percentage of cells expressing iEC markers in different conditions.

FIGS. 27A-27B depict addition of Angiopoietin-1 to enhance expression of CD144 marker by day 21 (27A) protein expression of endothelial markers CD144 at day 21 (27B) percentage of cells expressing iEC markers in different conditions.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006), and Sambrook and Russel, Molecular Cloning: A Laboratory Manual 4^{th} ed, Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

As used herein the phrase “glucose responsive” refers to a cell's ability to secret insulin when challenged to a glucose stimulation assay.

As used herein the term “reproducible” when used in conjunction with various methods of differentiations described herein refers to a method that is successful in at least three independent rounds of differentiations.

As used herein “MATRIGEL” refers to the solubilized basement membrane matrix secreted by Engelbreth-Holm-Swarm (EHS) mouse sarcoma cells; for example, produced by Corning Life Sciences.

As used herein, a “subject” means a human or animal. Usually the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal may be a human, non-human primate, mouse, rat, dog, cat, horse, or cow, but are not limited to these examples. In addition, the methods described herein may be used to treat domesticated animals and/or pets. In some embodiments, the subject is a human.

A subject may be one who has been previously diagnosed with or identified as suffering from or having a disease, disorder or condition in need of treatment or one or more complications related to the disease, disorder, or condition, and optionally, have already undergone treatment for the disease, disorder, or condition or the one or more complications related to the disease, disorder, or condition. Alternatively, a subject can also be one who has not been previously diagnosed as having a disease, disorder, or condition or one or more complications related to the disease, disorder, or condition. For example, a subject may be one who exhibits one or more risk factors for a disease, disorder, or condition or one or more complications related to the disease, disorder, or condition or a subject who does not exhibit risk factors. A “subject in need” of treatment for a particular disease, disorder, or condition may be a subject suspected of having that disease, disorder, or condition, diagnosed as having that disease, disorder, or condition, already treated or being treated for that disease, disorder, or condition, not treated for that disease, disorder, or condition, or at risk of developing that disease, disorder, or condition.

The inventors describe herein, among other things, an isogenic co-culture method using iPSC-derived pancreatic islets (iIslets) and endothelial cells (iECs) from the same donor, which lead to better maturation and functionality of iIslets with higher expression of β-cell markers and insulin secretion synchronized with high glucose challenges. This is the first time for the Inventors' knowledge that iIslets and iECs were generated from the same iPSC donors and their co-culture resulted in remarkably more defined and functional β-cells.

Human induced pluripotent stem cells (iPSCs) are derived from adult somatic cells such as cells from the skin or blood that have been genetically reprogrammed to an embryonic stem cell-like state, giving them the ability to grow indefinitely (self-renewal) and to give rise to any desired cell of the body from the three germ layers using specific cocktails of small molecules, transcription factors and growth factors, making iPSCs a source for the generation of an endless number of differentiated cells. Several have used human embryonic stem cells (ESCs) that are obtained from discarded embryos, which impose some ethical issues. Aside from ethical limitations, β-cell therapies derived from a single allogenic cell source such as an ESC line are likely to become refractory to the recipient because of alloimmunization against human leukocyte antigens (HLAs). To circumvent the immunogenicity that can be caused by β-cells derived from ESCs, utilization of patient iPSC-derived β-cells from their own blood seems to be a solution. Alternatively, using an iPSC haplobank populated with the most frequent homozygous HLA haplotype donors, selected for maximum utility to match the intended recipient U.S. population, allows for scalability of such an approach. In addition, many of the strategies generating β-cells from ESCs give rise to polyhormonal cells, i.e. insulin⁺/glucagon⁺/somatostatin⁺ cells that cannot retain a monohormonal insulin⁺ state, besides having a low glucose threshold for insulin secretion in vitro, which is amplified only several weeks after transplantation in vivo, when these cells acquire a more mature profile. Thus, complex signals are likely key for the maturation of β-cells in vivo, and testing these signals in vitro is urgent to develop improved protocols.

Some signals that are found to be crucial for the development and maturation of the endocrine pancreas are provided by surrounding vascular endothelial cells (ECs), and these signals are not included in previous protocols for β-cell differentiation from stem cells. ECs are part of the vasculature and are now considered as an active organ that is critical to the function of the vasculature as well as function of organs throughout the body. Particularly in the endocrine pancreas, a high vascular blood supply is needed by the islets because of their vigorous active role in maintaining glycemia through sensing external signals such as glucose levels and secreting hormones such as insulin, glucagon and somatostatin. Although the knowledge on this field has evolved, the exact mechanisms and pathways underlying the interaction between ECs and the endocrine pancreas had not been fully understood and the Inventors believe they are key to the development and maturation of iPSC-derived β-cells in vitro, which has been explored in the Inventors' work as further described herein.

Various embodiments of the present invention provide for a method of generating functional induced pluripotent stem cell (iPSC) derived pancreatic islets (iIslets), comprising: co-culturing a quantity of iPSC derived vascular endothelial cells (iECs) and a quantity of iPSC derived islet progenitors for about 10-18 days to generate the functional iIslets comprising β-cells.

In various embodiments, co-culturing comprises: plating a quantity of iPSC derived vascular endothelial cells (iECs) on MATRIGEL-coated plates and culturing in Phase IV EC media supplemented with Y27632; plating a quantity of iPSC derived pancreatic islets (iIslets) on top of the quantity of iECs and either culturing in media comprising about ½ Phase IV iEC media and about ½ Phase VI islet media supplemented with Y-27632 for about 12-16 days, or culturing in Phase VI islet media (islet only condition) for about 12-16 days to generate the functional iIslets comprising β-cells. In various embodiments, the concentration of Y27632 is about 10 μM. In various embodiments, the concentration of Y27632 is about 8-12 μM.

In various embodiments, co-culturing comprises: plating a quantity of iPSC derived vascular endothelial cells (iECs) on MATRIGEL-coated plates and culturing in Phase IV EC media supplemented with Y27632; plating a quantity of iPSC derived pancreatic islets (iIslets) on top of the quantity of iECs and either culturing in media comprising about ½ Phase IV iEC media and about ½ Phase VI islet media supplemented with Y-27632 for about 14 days, or culturing in Phase VI islet media (islet only condition) for about 14 days to generate the functional iIslets comprising β-cells.

In various embodiments, the method comprises generating the iECs before co-culturing, wherein the iECs are generated by plating a quantity of induced pluripotent stem cells (iPSCs) onto MATRIGEL; culturing the iPSC in MATRIGEL for about 2-4 days; culturing in the presence of CHIR99021 for about 1-3 days to generate mesoderm; culturing the mesoderm in the presence of BMP4, FGF2, and VEGF for about 1-3 days to generate vascular progenitors; and culturing the vascular progenitors in the presence of VEGF and Y-27632 for about 3-8 days to generate endothelial cell (EC) progenitors.

In various embodiments, the method comprises generating the iECs before co-culturing, wherein the iECs are generated by plating a quantity of induced pluripotent stem cells (iPSCs) onto MATRIGEL; culturing the iPSC in MATRIGEL for about 3 days; culturing in the presence of CHIR99021 for about 2 days to generate mesoderm; culturing the mesoderm in the presence of BMP4, FGF2, and VEGF for about 2 days to generate vascular progenitors; and culturing the vascular progenitors in the presence of VEGF and Y-27632 for about 4-7 days to generate endothelial cell (EC) progenitors.

In various embodiments for generating the iECs, the iPSCs are planted in planar on the MATRIGEL-coated plates as small colonies of cells. In various embodiments, the concentration of CHIR99021 is about 4-8 μM. In various embodiments, the concentration of CHIR99021 is about 6 μM. In various embodiments, the concentrations of BMP4, FGF2, and VEGF are about 25 ng/ml (BMP4), about 10 ng/ml (FGF2) and about 50 ng/ml (VEGF). In various embodiments, the concentrations of BMP4, FGF2, and VEGF are about 20-30 ng/ml (BMP4), about 8-12 ng/ml (FGF2) and about 40-60 ng/ml (VEGF). In various embodiments, the media is changed about every other day. In various embodiments, the media is changed about every day. In various embodiments, the media is changed about every two days.

In various embodiments, the method comprises generating the quantity of islet progenitors before co-culturing, wherein the islet progenitors are generated by culturing a quantity of induced pluripotent stem cells (iPSCs) in the presence of Activin-A, CHIR99021 and ROCK inhibitor (e.g.,) Y-27632 for about 1-2 days; culturing in the presence of Activin-A and FGF2 for about 1-3 days; culturing in the presence of FGF10, CHIR99021 and Noggin for about 1-3 days, to generate posterior foregut cells; culturing the posterior foregut cells in the presence of FGF10, Noggin, RA and SANT1 for about 3-5 days to generate pancreatic progenitors; culturing the pancreatic progenitors in the presence of Noggin, EGF and Nicotinamide for about 3-5 days to generate pancreatic endocrine progenitors; culturing the pancreatic endocrine progenitors in the presence of Noggin, T3 and Alk5i II for about 6-8 days to generate islet progenitors.

In various embodiments, the method comprises generating the quantity of islet progenitors before co-culturing, wherein the islet progenitors are generated by culturing a quantity of induced pluripotent stem cells (iPSCs) in the presence of Activin-A, CHIR99021 and ROCK inhibitor (e.g., Y-27632) for about 1 day; culturing in the presence of Activin-A and FGF2 for about 2 days; culturing in the presence of FGF10, CHIR99021 and Noggin for about 2 days, to generate posterior foregut cells; culturing the posterior foregut cells in the presence of FGF10, Noggin, RA and SANT1 for about 4 days to generate pancreatic progenitors; culturing the pancreatic progenitors in the presence of Noggin, EGF and Nicotinamide for about 4 days to generate pancreatic endocrine progenitors; culturing the pancreatic endocrine progenitors in the presence of Noggin, T3 and Alk5i II for about 7 days to generate islet progenitors.

In various embodiments for generating the quantity of islet progenitors, the iPSCs are single cell dissociated and re-seeded with ROCK inhibitor (e.g., Y-27632) and planted in planar on the MATRIGEL-coated plates at a density of about 300,000 cells/cm². In various embodiments, the density is about 250,000-350,000 cells/cm². In various embodiments, the concentration of the ROCK inhibitor is about 10 mM. In various embodiments, the concentration of the ROCK inhibitor is about 8-12 mM. In various embodiments, the concentrations of Activin-A, CHIR99021, and Y-27632 are about 80-120 ng/ml (Activin-A), about 1-3 μM CHIR99021 and about 8-12 μM (Y-27632). In various embodiments, the concentrations of Activin-A, CHIR99021, and Y-27632 are about 100 ng/ml (Activin-A), about 2 μM CHIR99021 and about 10 μM (Y-27632). In various embodiments, the concentrations of Activin-A and FGF2 are about 80-120 ng/ml (Activin-A) and about 4-6 ng/ml FGF2 (FGF2). In various embodiments, the concentrations of Activin-A and FGF2 are about 100 ng/ml (Activin-A) and about 5 ng/ml FGF2 (FGF2). In various embodiments, the concentrations of FGF10, Noggin, RA and SANT1 are about 40-60 ng/ml FGF10, about 40-60 ng/ml Noggin, about 1-3 μM RA and about 0.2-0.3 μM SANT1. In various embodiments, the concentrations of FGF10, Noggin, RA and SANT1 are about 50 ng/ml FGF10, about 50 ng/ml Noggin, about 2 μM RA and about 0.25 μM SANT1. In various embodiments, the concentrations of Noggin, EGF and Nicotinamide are about 40-60 ng/ml Noggin, about 80-120 ng/ml EGF and about 8-12 mM Nicotinamide. In various embodiments, the concentrations of Noggin, EGF and Nicotinamide are about 50 ng/ml Noggin, about 100 ng/ml EGF and about 10 mM Nicotinamide. In various embodiments, the concentrations of Noggin, T3 and Alk5i II are about 40-60 ng/ml Noggin, about 0.5-1.5 μM T3 and about 8-10 μM Alk5i II. In various embodiments, the concentrations of Noggin, T3 and Alk5i II are about 50 ng/ml Noggin, about 1 μM T3 and about 10 μM Alk5i II. In various embodiments, the media is changed about every other day. In various embodiments, the media is changed about every day. In various embodiments, the media is changed about every two days.

In various embodiments, the pancreatic progenitors express PDX1+ and SOX9+.

In various embodiments, the pancreatic endocrine progenitors are PDX1+ and NKX6.1⁺.

In various embodiments, the iIslets express C-peptide, glucagon and NKX6.1⁺.

In various embodiments, the expression of INS, UCN3, NGN3 and CHGA are upregulated in the β-cell that are produced in the islets only condition, as compared to β-cell that are produced without co-culturing with vascular endothelial cells or as compared to β-cell that were produced in a culture without the islets only condition.

In various embodiments, the β-cell increase insulin secretion when challenged with a high glucose concentration as compared to a basal glucose concentration. In various embodiments, the β-cell increase insulin secretion by at least 50% when challenged with a high glucose concentration as compared to a basal glucose concentration. In various embodiments, the β-cell increase insulin secretion by at least 100% when challenged with a high glucose concentration as compared to a basal glucose concentration. In various embodiments, the β-cell increase insulin secretion by at least 200% when challenged with a high glucose concentration as compared to a basal glucose concentration. In various embodiments, the β-cell increase insulin secretion by at least 300% when challenged with a high glucose concentration as compared to a basal glucose concentration. In various embodiments, the β-cell increase insulin secretion by at least 400% when challenged with a high glucose concentration as compared to a basal glucose concentration. In various embodiments, the β-cell increase insulin secretion by at least 500% when challenged with a high glucose concentration as compared to a basal glucose concentration.

In various embodiments, the iPSC derived vascular endothelial cells (iECs) and iPSC derived islet progenitors are isogenic.

In various embodiments, the iPSCs used to derive vascular endothelial cells (iECs) and iPSC used to derive islet progenitors are from the same iPSC cell line or from the same donor.

In various embodiments, the iPSCs used to derive vascular endothelial cells (iECs) and iPSC used to derive islet progenitors are generated from a subject who will receive the iIslets. That is, the source cells for the iPSCs and the generated iIslets are personalized for the same person.

In various embodiments, the iIslets are human iIslets.

Various embodiments of the present invention provide for iIslets generated by any one of the methods of the present invention as described herein.

Various embodiments of the present invention provide for iIslets. In various embodiments, the iIslets express C-peptide, glucagon and NKX6.1+. In various embodiments, the iIslets increase insulin secretion when challenged with a high glucose concentration as compared to a basal glucose concentration.

In various embodiments, the iIslets are from a composition of comprising iIslets generated by a method of the present invention as described herein. For example, inventive methods of the present invention are used to generate the iIslets and the iIslets are then frozen for storage. In another example, inventive methods of the present invention are used to generate the iIslets, and the iIslets are passaged multiple times. Thus, those passaged iIslets are in a composition of comprising iIslets generated by a method of the present invention as described herein even if they are not directly generated by the inventive methods described herein.

Various embodiments of the present invention provide a method of ameliorating or treating a metabolic disease, metabolic disorder or metabolic condition in a subject in need thereof, comprising: administering iIslets of the present invention to the subject in need thereof to ameliorate or treat the condition. In various embodiments, the metabolic disease, metabolic disorder or metabolic condition is diabetes or insulin resistance.

Various embodiments of the present invention provide a method of administering iIslets of the present invention to a subject in need thereof, comprising: administering iIslets of the present invention to the subject in need thereof, wherein the subject is in need of amelioration or treatment of a metabolic disease, metabolic disorder or metabolic condition. In various embodiments, the metabolic disease, metabolic disorder or metabolic condition is diabetes or insulin resistance.

Various embodiments described herein provide for a method of cellular differentiation, including providing a quantity of induced pluripotent stem cells (iPSCs), culturing in the presence of Activin-A, CHIR99021 and Y-27632 for about 1 day, further culturing in the presence of Activin-A and FGF2 for about 2 days, additionally culturing in the presence of FGF10, CHIR99021 and Noggin for about 2 days, to generate posterior foregut. In other embodiments, the method includes culturing the posterior foregut in the presence of FGF10, Noggin, RA and SANT1 for about 4 days to generate pancreatic progenitors. In other embodiments, the pancreatic progenitors express one or more of PDX1⁺ and SOX9⁺. In other embodiments, the pancreatic progenitors express PDX1⁺ and SOX9⁺. In other embodiments, the method includes culturing the pancreatic progenitors in the presence of Noggin, EGF and Nicotinamide for about 4 days to generate pancreatic endocrine progenitors. In other embodiments, the pancreatic endocrine progenitors express one or more of PDX1⁺ and NKX6.1⁺. In other embodiments, the pancreatic endocrine progenitors are PDX1⁺ and NKX6.1⁺. In other embodiments, the method includes culturing the pancreatic endocrine progenitors in the presence of Noggin, T3 and Alk5i II for about 7 days to generate islet progenitors. In some embodiments, the media is changed every other day. In other embodiments, the method includes culturing the generate islet progenitors in the presence of T3, Alk5i II, R428, and N-acetylcysteine (NAC) for about 14 days to generate mature islets. In some embodiments, the media is changed every other day. In other embodiments, the mature islets express one or more of C-peptide, glucagon and NKX6.1⁺. In other embodiments, the mature islets express C-peptide, glucagon and NKX6.1⁺. In other embodiments, the mature islets express one or more of insulin (INS), urocortin-3 (UCN3), neurogenin-3 (NGN3) and chromogranin-A (CHGA). In other embodiments, the mature islets express insulin (INS), urocortin-3 (UCN3), neurogenin-3 (NGN3) and chromogranin-A (CHGA). In various embodiments, the islet progenitors are polyhormonal. In various embodiments, the mature islets are monohormonal. For example, polyhormonal cells may be insulin⁺/glucagon⁺/somatostatin⁺. In contrast, monohormonal cells are C-peptide⁺/glucagon⁻. In various embodiments, the mature islets secrete insulin and/or are glucose responsive. Each of the aforementioned growth factors are added at a concentration of 0.25 ng/ml to 250 ng/ml, small molecules are added at 0.1 μM to 2.5 mM. In various embodiments, each of the aforementioned growth factors are added at a concentration of about 0.25 ng/ml to 1 ng/ml, or 1 ng/ml to 10 ng/ml, or 10 ng/ml to 100 ng/ml, or 100 ng/ml to 200 ng/ml, or 200 ng/ml to 300 ng/ml. In various embodiments, each of the aforementioned small molecules are added at a concentration of about 0.1 μM to 1 μM, or 1 μM to 10 μM, or 10 μM to 50 μM, or 50 μM to 100 μM, or 100 μM to 1 mM, or 1 mM to 5 mM,

For example, iPSCs (OCT4 expression >90%) maintained on MATRIGEL-coated plates were single cell dissociated with Accutase and re-seeded with ROCK inhibitor Y-27632 (10 mM, R&D Systems) in planar onto MATRIGEL-coated plates at a density of 300,000 cells/cm² with mTeSR⁺ medium. 24-hr later, iPSCs were directed to definitive endoderm (DE—Phase I) using a combination of Activin-A (100 ng/ml, R&D Systems), CHIR99021 (2 μM, XcessBio) and Y-27632 (10 μM) for 1 day, followed by Activin-A (100 ng/ml) and FGF2 (5 ng/ml, PeproTech) for 2 days. Next, for the patterning of posterior foregut (PFG—Phase II), a combination of FGF10 (50 ng/ml, PeproTech), CHIR99021 (0.25 μM) and Noggin (50 ng/ml, PeproTech) was used for 2 days. To direct cells towards PDX1⁺/SOX9⁺ pancreatic progenitors (PP—Phase III), a combination of FGF10 (50 ng/ml), Noggin (50 ng/ml), RA (2 μM, Cayman) and SANT1 (0.25 μM, Sigma) was used for 4 days. Later, PDX1⁺/NKX6.1⁺ pancreatic endocrine progenitors (PEP—Phase IV) were induced through treatment with Noggin (50 ng/ml), EGF (100 ng/ml, PeproTech) and Nicotinamide (10 mM, Sigma) for 4 days. For the generation of islet progenitors (IP—Phase V), a combination of Noggin (50 ng/ml), T3 (1 μM, Sigma) and Alk5i II (10 μM, Axxora) was used for 7 days with media changes every other day. For the maturation of islets (MI—Phase VI), a combination of T3 (1 μM), Alk5i II (10 μM), AXL inhibitor R428 (2 Mm, Selleckchem) and antioxidant N-acetylcysteine NAC (1 mM, Sigma) was used for 14 days with media changes every other day.

In various embodiments, the iPSCs are single cell dissociated and re-seeded with ROCK inhibitor (e.g., Y-27632) and planted in planar on the MATRIGEL-coated plates at a density of about 300,000 cells/cm². In various embodiments, the density is about 250,000-350,000 cells/cm². In various embodiments, the concentration of the ROCK inhibitor is about 10 mM. In various embodiments, the concentration of the ROCK inhibitor is about 8-12 mM. In various embodiments, the concentrations of Activin-A, CHIR99021, and Y-27632 are about 80-120 ng/ml (Activin-A), about 1-3 μM CHIR99021 and about 8-12 μM (Y-27632). In various embodiments, the concentrations of Activin-A, CHIR99021, and Y-27632 are about 100 ng/ml (Activin-A), about 2 μM CHIR99021 and about 10 μM (Y-27632). In various embodiments, the concentrations of Activin-A and FGF2 are about 80-120 ng/ml (Activin-A) and about 4-6 ng/ml FGF2 (FGF2). In various embodiments, the concentrations of Activin-A and FGF2 are about 100 ng/ml (Activin-A) and about 5 ng/ml FGF2 (FGF2). In various embodiments, the concentrations of FGF10, Noggin, RA and SANT1 are about 40-60 ng/ml FGF10, about 40-60 ng/ml Noggin, about 1-3 μM RA and about 0.2-0.3 μM SANT1. In various embodiments, the concentrations of FGF10, Noggin, RA and SANT1 are about 50 ng/ml FGF10, about 50 ng/ml Noggin, about 2 μM RA and about 0.25 μM SANT1. In various embodiments, the concentrations of Noggin, EGF and Nicotinamide are about 40-60 ng/ml Noggin, about 80-120 ng/ml EGF and about 8-12 mM Nicotinamide. In various embodiments, the concentrations of Noggin, EGF and Nicotinamide are about 50 ng/ml Noggin, about 100 ng/ml EGF and about 10 mM Nicotinamide. In various embodiments, the concentrations of Noggin, T3 and Alk5i II are about 40-60 ng/ml Noggin, about 0.5-1.5 μM T3 and about 8-10 μM Alk5i II. In various embodiments, the concentrations of Noggin, T3 and Alk5i II are about 50 ng/ml Noggin, about 1 μM T3 and about 10 μM Alk5i II. In various embodiments, the media is changed about every other day. In various embodiments, the media is changed about every day. In various embodiments, the media is changed about every two days.

Various embodiments described herein provide for a quantity of induced pluripotent stem cells (iPSC) derived mature islets. In other embodiments, the mature islets express one or more of C-peptide, glucagon and NKX6.1⁺. In other embodiments, the mature islets express C-peptide, glucagon and NKX6.1⁺. In other embodiments, the mature islets express one or more of insulin (INS), urocortin-3 (UCN3), neurogenin-3 (NGN3) and chromogranin-A (CHGA). In other embodiments, the mature islets express insulin (INS), urocortin-3 (UCN3), neurogenin-3 (NGN3) and chromogranin-A (CHGA). In various embodiments, the islet progenitors are polyhormonal. In various embodiments, the mature islets are monohormonal. For example, polyhormonal cells may be insulin⁺/glucagon⁺/somatostatin⁺. In contrast, monohormonal cells are C-peptide⁺/glucagon⁺. In various embodiments, the mature islets secrete insulin and/or are glucose responsive. In various embodiment, the mature islet cells are glucose responsive in a glucose stimulating insulin secretion (GSIS) assay. In various embodiments, the GSIS assay is static. In various embodiments, the GSIS assay is dynamic. In various embodiments, the GSIS includes mature islet cells in an assembly with mature endothelial cells, organized as a vascularized channel, wherein the vascularized channel is capable of glucose challenge for the mature islet cells.

Various embodiments described herein provide for a quantity of mature islets made by the method, including providing a quantity of induced pluripotent stem cells (iPSCs), culturing in the presence of Activin-A, CHIR99021 and Y-27632 for about 1 day, further culturing in the presence of Activin-A and FGF2 for about 2 days, additionally culturing in the presence of FGF10, CHIR99021 and Noggin for about 2 days, to generate posterior foregut. In other embodiments, the method includes culturing the posterior foregut in the presence of FGF10, Noggin, RA and SANT1 for about 4 days to generate pancreatic progenitors. In other embodiments, the pancreatic progenitors express one or more of PDX1⁺ and SOX9⁺. In other embodiments, the pancreatic progenitors express PDX1⁺ and SOX9⁺. In other embodiments, the method includes culturing the pancreatic progenitors in the presence of Noggin, EGF and Nicotinamide for about 4 days to generate pancreatic endocrine progenitors. In other embodiments, the pancreatic endocrine progenitors express one or more of PDX1⁺ and NKX6.1⁺. In other embodiments, the pancreatic endocrine progenitors are PDX1⁺ and NKX6.1⁺. In other embodiments, the method includes culturing the pancreatic endocrine progenitors in the presence of Noggin, T3 and Alk5i II for about 7 days to generate islet progenitors. In some embodiments, the media is changed every other day. In other embodiments, the method includes culturing the generate islet progenitors in the presence of T3, Alk5i II, R428, and N-acetylcysteine (NAC) for about 14 days to generate mature islets. In some embodiments, the media is changed every other day. In other embodiments, the mature islets express one or more of C-peptide, glucagon and NKX6.1⁺. In other embodiments, the mature islets express C-peptide, glucagon and NKX6.1⁺. In other embodiments, the mature islets express one or more of insulin (INS), urocortin-3 (UCN3), neurogenin-3 (NGN3) and chromogranin-A (CHGA). In other embodiments, the mature islets express two or more of insulin (INS), urocortin-3 (UCN3), neurogenin-3 (NGN3) and chromogranin-A (CHGA). In other embodiments, the mature islets express three or more of insulin (INS), urocortin-3 (UCN3), neurogenin-3 (NGN3) and chromogranin-A (CHGA). In other embodiments, the mature islets express insulin (INS), urocortin-3 (UCN3), neurogenin-3 (NGN3) and chromogranin-A (CHGA). In various embodiments, the islet progenitors are polyhormonal. In various embodiments, the mature islets are monohormonal. For example, polyhormonal cells may be insulin⁺/glucagon⁺/somatostatin⁺. In contrast, monohormonal cells are C-peptide⁺/glucagon⁺. In various embodiments, the mature islets secrete insulin and/or are glucose responsive. In various embodiment, the mature islet cells are glucose responsive in a glucose stimulating insulin secretion (GSIS) assay. In various embodiments, the GSIS assay is static. In various embodiments, the GSIS assay is dynamic. In various embodiments, the GSIS includes mature islet cells in an assembly with mature endothelial cells, organized as a vascularized channel, wherein the vascularized channel is capable of glucose challenge for the mature islet cells.

Various embodiments described herein provide for a method of cellular differentiation, including providing a quantity of induced pluripotent stem cells (iPSCs), plating the iPSCs onto MATRIGEL, culturing for about iPSC in MATRIGEL for about 3 days, further culturing in the presence of CHIR99021 to generate mesoderm. In other embodiments, the method includes culturing the mesoderm in the presence of BMP4, FGF2, and VEGF for about 2 days to generate vascular progenitors.

In various embodiments, the iPSCs are planted in planar on the MATRIGEL-coated plates as small colonies of cells. In various embodiments, the concentration of CHIR99021 is about 4-8 μM. In various embodiments, the concentration of CHIR99021 is about 6 μM. In various embodiments, the concentrations of BMP4, FGF2, and VEGF are about 25 ng/ml (BMP4), about 10 ng/ml (FGF2) and about 50 ng/ml (VEGF). In various embodiments, the concentrations of BMP4, FGF2, and VEGF are about 20-30 ng/ml (BMP4), about 8-12 ng/ml (FGF2) and about 40-60 ng/ml (VEGF). In various embodiments, the media is changed about every other day. In various embodiments, the media is changed about every day. In various embodiments, the media is changed about every two days.

In some embodiments, the vascular progenitors are dissociated and replated at a density of about 85,000-100,000 cells/cm². In some embodiments, the vascular progenitors are dissociated and replated at a density of about 85,000 cells/cm², or about 85,000 cells/cm², or about 90,000 cells/cm², or about 95,000 cells/cm², or about 100,000 cells/cm², or about 105,000 cells/cm². In other embodiments, the method includes culturing the vascular progenitors in the presence of VEGF and Y-27632 for about 7 days to generate endothelial cell (EC) progenitors. In some embodiments, the media is changed every other day. In other embodiments, the EC progenitors are cultured for an additional about 3 days. In other embodiments, the EC progenitors are dissociated and replated onto MATRIGEL-coated plates. In other embodiments, the method includes culturing the EC progenitors with VEGF for about 10 days to generate mature EC. In some embodiments, the mature EC are replated onto MATRIGEL-coated plates. In other embodiments, the method includes culturing the mature EC with VEGF for about 10 days. In other embodiments, vascular progenitors and endothelial progenitors are cultured in the presence of Angiopoietin-1. In various embodiments, vascular progenitors and endothelial progenitors are cultured in the presence of Angiopoietin-1 from day 4 to day 21 of differentiation. In other embodiments, the method includes culturing vascular progenitors in the presence of EGM-MV2 and VEGF for about 4-6 days to mature generate endothelial progenitor cells, and culturing endothelial progenitor cells in the presence of EGM-MV2 and VEGF to generate mature endothelial cells. In some embodiments, the media is changed every other day. In other embodiments, the mature EC express one or more of CD31⁺, CD144⁺, VEGF-A⁺, VEGFR2⁺, and Ac-LDL. In other embodiments, the mature EC express CD31⁺, CD144⁺, VEGF-A⁺, VEGFR2⁺, and Ac-LDL. Each of the aforementioned growth factors are added at a concentration of 0.25 ng/ml to 250 ng/ml, small molecules are added at 0.1 μM to 2.5 mM.

For example, for the generation of pancreatic islets from iPSCs (iIslets), the Inventors have developed a robust and reproducible protocol. Briefly, iPSCs (OCT4 expression >90%) maintained on MATRIGEL-coated plates were single cell dissociated with Accutase and re-seeded with ROCK inhibitor Y-27632 (10 mM, R&D Systems) in planar onto MATRIGEL-coated plates at a density of 300,000 cells/cm² with mTeSR⁺ medium. 24-hr later, iPSCs were directed to definitive endoderm (DE—Phase I) using a combination of Activin-A (100 ng/ml, R&D Systems), CHIR99021 (2 μM, XcessBio) and Y-27632 (10 μM) for 1 day, followed by Activin-A (100 ng/ml) and FGF2 (5 ng/ml, PeproTech) for 2 days. Next, for the patterning of posterior foregut (PFG—Phase II), a combination of FGF10 (50 ng/ml, PeproTech), CHIR99021 (0.25 μM) and Noggin (50 ng/ml, PeproTech) was used for 2 days. To direct cells towards PDX1⁺/SOX9⁺ pancreatic progenitors (PP—Phase III), a combination of FGF10 (50 ng/ml), Noggin (50 ng/ml), RA (2 μM, Cayman) and SANT1 (0.25 μM, Sigma) was used for 4 days. Later, PDX1⁺/NKX6.1⁺ pancreatic endocrine progenitors (PEP—Phase IV) were induced through treatment with Noggin (50 ng/ml), EGF (100 ng/ml, PeproTech) and Nicotinamide (10 mM, Sigma) for 4 days. For the generation of islet progenitors (IP—Phase V), a combination of Noggin (50 ng/ml), T3 (1 μM, Sigma) and Alk5i II (10 μM, Axxora) was used for 7 days with media changes every other day. For the maturation of islets (MI—Phase VI), a combination of T3 (1 μM), Alk5i II (10 μM), AXL inhibitor R428 (2 Mm, Selleckchem) and antioxidant N-acetylcysteine NAC (1 mM, Sigma) was used for 14 days with media changes every other day. The formulation of the base media used throughout the differentiation is summarized at Table 2.

Various embodiments described herein provide for a quantity of induced pluripotent stem cells (iPSC) derived mature endothelial cells (EC). In other embodiments, the mature EC express one or more of CD31⁺, CD144⁺, VEGF-A⁺, VEGFR2⁺, and Ac-LDL. In other embodiments, the mature EC express two or more of CD31⁺, CD144⁺, VEGF-A⁺, VEGFR2⁺, and Ac-LDL. In other embodiments, the mature EC express three or more of CD31⁺, CD144⁺, VEGF-A⁺, VEGFR2⁺, and Ac-LDL. In other embodiments, the mature EC express four or more of CD31⁺, CD144⁺, VEGF-A⁺, VEGFR2⁺, and Ac-LDL. In other embodiments, the mature EC express CD31⁺, CD144⁺, VEGF-A⁺, VEGFR2⁺, and Ac-LDL.

Various embodiments described herein provide for a quantity of mature endothelial cells (EC) made by the method, including providing a quantity of induced pluripotent stem cells (iPSCs), plating the iPSCs onto MATRIGEL, culturing for about iPSC in MATRIGEL for about 3 days, further culturing in the presence of CHIR99021 to generate mesoderm. In other embodiments, the method includes culturing the mesoderm in the presence of BMP4, FGF2, and VEGF for about 2 days to generate vascular progenitors. In other embodiments, the method includes culturing the vascular progenitors in the presence of VEGF and Y-27632 for about 7 days to generate endothelial cell (EC) progenitors. In some embodiments, the media is changed every other day. In other embodiments, vascular progenitors are enzymatically dissociated (e.g., Accutase) and are re-plated in MATRIGEL-coated plates at a density of 85,000-100,000 cells/cm² to generate EC progenitors. In other embodiments, the method includes culturing the EC progenitors with VEGF for about 10 days to generate mature EC. In other embodiments, vascular progenitors and endothelial progenitors are cultured in the presence of Angiopoietin-1. In various embodiments, vascular progenitors and endothelial progenitors are cultured in the presence of Angiopoietin-1 from day 4 to day 21 of differentiation. In other embodiments, the method includes culturing vascular progenitors in the presence of EGM-MV2 and VEGF for about 4-6 days to generate mature endothelial progenitor cells, and culturing endothelial progenitor cells in the presence of EGM-MV2 and VEGF to generate mature endothelial cells. In some embodiments, the media is changed every other day. In other embodiments, the mature EC express one or more of CD31⁺, CD144⁺, VEGF-A⁺, VEGFR2⁺, and Ac-LDL. In other embodiments, the mature EC express two or more of CD31⁺, CD144⁺, VEGF-A⁺, VEGFR2⁺, and Ac-LDL. In other embodiments, the mature EC express three or more of CD31⁺, CD144⁺, VEGF-A⁺, VEGFR2⁺, and Ac-LDL. In other embodiments, the mature EC express four or more of CD31⁺, CD144⁺, VEGF-A⁺, VEGFR2⁺, and Ac-LDL. In other embodiments, the mature EC express CD31⁺, CD144⁺, VEGF-A⁺, VEGFR2⁺, and Ac-LDL.

Various embodiments described herein provide for an assembly, including a quantity of mature islets and a quantity of mature EC. In other embodiments, the mature islets and mature EC are isogenic. In some embodiments, the assembly further comprises a construct such as a scaffold. In various embodiments, the mature islets, mature EC, or both, are deposited on the scaffold. In various embodiments, the mature islets, mature EC or both, are deposited on the scaffold using a bioink. In various embodiments, the bioink includes fibrin or alginate.

In other embodiments, the mature islets are made by the method, including providing a quantity of induced pluripotent stem cells (iPSCs), culturing in the presence of Activin-A, CHIR99021 and Y-27632 for about 1 day, further culturing in the presence of Activin-A and FGF2 for about 2 days, additionally culturing in the presence of FGF10, CHIR99021 and Noggin for about 2 days, to generate posterior foregut. In other embodiments, the method includes culturing the posterior foregut in the presence of FGF10, Noggin, RA and SANT1 for about 4 days to generate pancreatic progenitors. In other embodiments, the pancreatic progenitors express one or more of PDX1⁺ and SOX9⁺. In other embodiments, the pancreatic progenitors express PDX1⁺ and SOX9⁺. In other embodiments, the method includes culturing the pancreatic progenitors in the presence of Noggin, EGF and Nicotinamide for about 4 days to generate pancreatic endocrine progenitors. In other embodiments, the pancreatic endocrine progenitors express one or more of PDX1⁺ and NKX6.1⁺. In other embodiments, the pancreatic endocrine progenitors are PDX1⁺ and NKX6.1⁺. In other embodiments, the method includes culturing the pancreatic endocrine progenitors in the presence of Noggin, T3 and Alk5i II for about 7 days to generate islet progenitors. In other embodiments, the method includes culturing the generate islet progenitors in the presence of T3, Alk5i II, R428, and N-acetylcysteine (NAC) for about 14 days to generate mature islets. In other embodiments, the mature islets express one or more of C-peptide, glucagon and NKX6.1⁺. In other embodiments, the mature islets express C-peptide, glucagon and NKX6.1⁺. In other embodiments, the mature islets express one or more of insulin (INS), urocortin-3 (UCN3), neurogenin-3 (NGN3) and chromogranin-A (CHGA). In other embodiments, the mature islets express insulin (INS), urocortin-3 (UCN3), neurogenin-3 (NGN3) and chromogranin-A (CHGA).

In other embodiments, the mature EC are made by the method, including providing a quantity of induced pluripotent stem cells (iPSCs), plating the iPSCs onto MATRIGEL, culturing for about iPSC in MATRIGEL for about 3 days, further culturing in the presence of CHIR99021 to generate mesoderm. In other embodiments, the method includes culturing the mesoderm in the presence of BMP4, FGF2, and VEGF for about 2 days to generate vascular progenitors. In other embodiments, the method includes culturing the vascular progenitors in the presence of VEGF and Y-27632 for about 7 days to generate endothelial cell (EC) progenitors. In other embodiments, the method includes culturing the EC progenitors with VEGF for about 10 days to generate mature EC. In other embodiments, vascular progenitors and endothelial progenitors are cultured in the presence of Angiopoietin-1. In various embodiments, vascular progenitors and endothelial progenitors are cultured in the presence of Angiopoietin-1 from day 4 to day 21 of differentiation. In other embodiments, the method includes culturing vascular progenitors in the presence of EGM-MV2 and VEGF for about 4-6 days to generate mature endothelial progenitor cells, and culturing endothelial progenitor cells in the presence of EGM-MV2 and VEGF to generate mature endothelial cells. In other embodiments, the mature EC express one or more of CD31⁺, CD144⁺, VEGF-A⁺, VEGFR2⁺, and Ac-LDL. In other embodiments, the mature EC express two or more of CD31⁺, CD144⁺, VEGF-A⁺, VEGFR2⁺, and Ac-LDL. In other embodiments, the mature EC express three or more of CD31⁺, CD144⁺, VEGF-A⁺, VEGFR2⁺, and Ac-LDL. In other embodiments, the mature EC express four or more of CD31⁺, CD144⁺, VEGF-A⁺, VEGFR2⁺, and Ac-LDL. In other embodiments, the mature EC express CD31⁺, CD144⁺, VEGF-A⁺, VEGFR2⁺, and Ac-LDL.

Various embodiments described herein provide for a method of administering matures islets made by the method as described herein, endothelial cells made by the method as described herein, or both, to a subject in need thereof. In various embodiments, the subject is afflicted with a metabolic disease, disorder and/or condition. In various embodiments, the metabolic disorder and/or condition is diabetes, and/or insulin resistance. In various embodiments, the cells are induced pluripotent stem cell (iPSC) derived cells. In various embodiments, the islet cells, endothelial cells, or both, are isogenic relative to the subject. In various embodiments, the matures islets made by the method as described herein, endothelial cells made by the method as described herein, or both are capable of modulating the metabolic disease, disorder and/or condition. In various embodiments, the matures islets made by the method as described herein, endothelial cells made by the method as described herein, or both are capable of treating the metabolic disease, disorder and/or condition.

In other embodiments, the mature islets are made by the method, including providing a quantity of induced pluripotent stem cells (iPSCs), culturing in the presence of Activin-A, CHIR99021 and Y-27632 for about 1 day, further culturing in the presence of Activin-A and FGF2 for about 2 days, additionally culturing in the presence of FGF10, CHIR99021 and Noggin for about 2 days, to generate posterior foregut. In other embodiments, the method includes culturing the posterior foregut in the presence of FGF10, Noggin, RA and SANT1 for about 4 days to generate pancreatic progenitors. In other embodiments, the pancreatic progenitors express one or more of PDX1⁺ and SOX9⁺. In other embodiments, the pancreatic progenitors express PDX1⁺ and SOX9⁺. In other embodiments, the method includes culturing the pancreatic progenitors in the presence of Noggin, EGF and Nicotinamide for about 4 days to generate pancreatic endocrine progenitors. In other embodiments, the pancreatic endocrine progenitors express one or more of PDX1⁺ and NKX6.1⁺. In other embodiments, the pancreatic endocrine progenitors are PDX1⁺ and NKX6.1⁺. In other embodiments, the method includes culturing the pancreatic endocrine progenitors in the presence of Noggin, T3 and Alk5i II for about 7 days to generate islet progenitors. In other embodiments, the method includes culturing the generate islet progenitors in the presence of T3, Alk5i II, R428, and N-acetylcysteine (NAC) for about 14 days to generate mature islets. In other embodiments, the mature islets express one or more of C-peptide, glucagon and NKX6.1⁺. In other embodiments, the mature islets express C-peptide, glucagon and NKX6.1⁺. In other embodiments, the mature islets express one or more of insulin (INS), urocortin-3 (UCN3), neurogenin-3 (NGN3) and chromogranin-A (CHGA). In other embodiments, the mature islets express two or more of insulin (INS), urocortin-3 (UCN3), neurogenin-3 (NGN3) and chromogranin-A (CHGA). In other embodiments, the mature islets express three or more of insulin (INS), urocortin-3 (UCN3), neurogenin-3 (NGN3) and chromogranin-A (CHGA). In other embodiments, the mature islets express insulin (INS), urocortin-3 (UCN3), neurogenin-3 (NGN3) and chromogranin-A (CHGA).

In other embodiments, the mature EC are made by the method, including providing a quantity of induced pluripotent stem cells (iPSCs), plating the iPSCs onto MATRIGEL, culturing for about iPSC in MATRIGEL for about 3 days, further culturing in the presence of CHIR99021 to generate mesoderm. In other embodiments, the method includes culturing the mesoderm in the presence of BMP4, FGF2, and VEGF for about 2 days to generate vascular progenitors. In other embodiments, the method includes culturing the vascular progenitors in the presence of VEGF and Y-27632 for about 7 days to generate endothelial cell (EC) progenitors. In other embodiments, the method includes culturing the EC progenitors with VEGF for about 10 days to generate mature EC. In other embodiments, vascular progenitors and endothelial progenitors are cultured in the presence of Angiopoietin-1. In various embodiments, vascular progenitors and endothelial progenitors are cultured in the presence of Angiopoietin-1 from day 4 to day 21 of differentiation. In other embodiments, the method includes culturing vascular progenitors in the presence of EGM-MV2 and VEGF for about 4-6 days to generate mature endothelial progenitor cells, and culturing endothelial progenitor cells in the presence of EGM-MV2 and VEGF to generate mature endothelial cells. In other embodiments, the mature EC express one or more of CD31⁺, CD144⁺, VEGF-A⁺, VEGFR2⁺, and Ac-LDL. In other embodiments, the mature EC express two or more of CD31⁺, CD144⁺, VEGF-A⁺, VEGFR2⁺, and Ac-LDL. In other embodiments, the mature EC express three or more of CD31⁺, CD144⁺, VEGF-A⁺, VEGFR2⁺, and Ac-LDL. In other embodiments, the mature EC express four or more of CD31⁺, CD144⁺, VEGF-A⁺, VEGFR2⁺, and Ac-LDL. In other embodiments, the mature EC express CD31⁺, CD144⁺, VEGF-A⁺, VEGFR2⁺, and Ac-LDL. Further information is found in U.S. Prov. App. 62/647,548 and PCT App. No. PCT/US2019/023749, which are fully incorporated by reference herein.

### Example 1

Investigating the Contribution of Specific Cell Populations of hiPSC-Derived Vascular Endothelial Cells (iECs) and Pancreatic Islets (iIslets) in Enhancing Generation of Functional iIslets Derived In Vitro

The Inventors have successfully generated methods to make iIslet progenitors (PDX1⁺/NKX6.1⁺) and vascular iECs from multiple hiPSC lines. After generating these cells, the Inventors have tested different methods of co-culturing iIslet progenitors with iECs and discovered an optimal approach where iECs improved the secretion of insulin of iIslets when challenged with glucose. However, the precise cell composition and signals exchanged between the iECs and iIslets contributing to enhanced iIslet function have not yet been investigated. Thus, the Inventors explore the nature of cell composition and mechanisms differently expressed in iIslets co-cultured with iECs through deep single cell transcriptomic analyses using single-cell RNA sequencing (sc RNA-seq).

Without being bound by any particular theory, it is believed that since iIslets co-cultured with iECs have enhanced functionality compared to iIslets generated alone, the Inventors believe that a greater number of monohormonal β-cells in the iIslets co-cultured with iECs will be found and there will be an upregulation of pathways linked to the insulin secretion machinery.

### Ex Vivo Dynamic Perfusion Systems

To establish novel ex vivo dynamic perfusion systems to test functionality of human iIslets. The Inventors found iIslets co-cultured with iECs presented an increased insulin secretion when challenged to a high concentration of glucose compared to iIslets cultured alone using a static assay (GSIS). To better characterize iIslets functionality in a more physiological fashion ex vivo, the Inventors will assess functionality of iIslets using dynamic GSIS in a novel 3D bioprinted vascularized iIslet platform, which consists of a perfusion system where iIslets are dynamically challenged with glucose through an iEC vascularized channel.

Without being bound by any particular theory, it is believed that iIslets generated in co-culture with iECs in this novel ex vivo 3D bioprinted human vascularized iIslet platform will present high a higher insulin secretion in a dynamic assay.

This is the first time that iIslets have been co-cultured with isogenic iECs (from same donor) to obtain better maturity and functionality of iIslets in vitro and in ex vivo 3D bioprinted human vascularized iIslet system. Importantly, the Inventors will be able to show the mechanisms and pathways underlying this interaction using the cutting-edge tool sc RNA-seq.

### Significance

The fact that pancreatic islets and ECs are generated from the same hiPSC donor is a crucial approach because these cells altogether could be transplanted back in the same diabetic patient, which could minimize the immunoreaction of the transplantation. In addition, this application brings novelty to better understand the heterogeneity of cell populations and how specific cell types contribute to enhanced functionality of iIslets co-cultured with iECs along with pathways that might contribute to this phenotype, using sc RNAseq analyses. Notably, by combining the powerful iPSC and 3D bioprinting technologies the Inventors propose to develop a novel ex vivo 3D bioprinted human vascularized (using iECs) iIslet prototype to better characterize the functionality of iIslets, which would closely mimic β-cell physiology with regard to insulin secretion in vivo.

### Experimental Design

The overall goal is to understand the nature of cell composition and mechanisms differently expressed in iIslets co-cultured with iECs, as well as establish a novel 3D bioprinted vascularized iIslet platform to better characterize iIslets function that closely mimics human physiology.

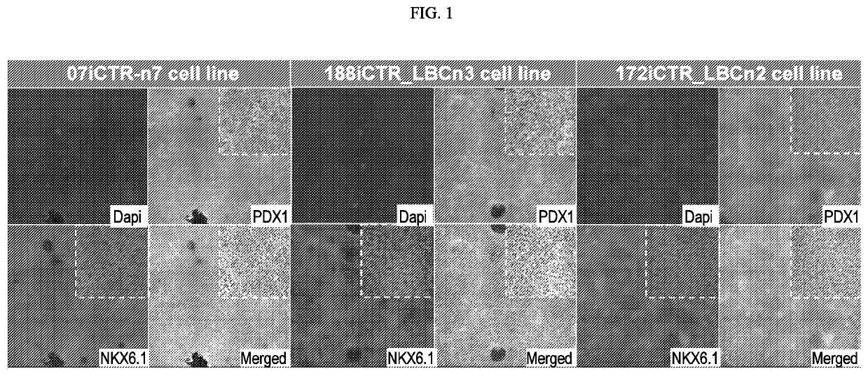

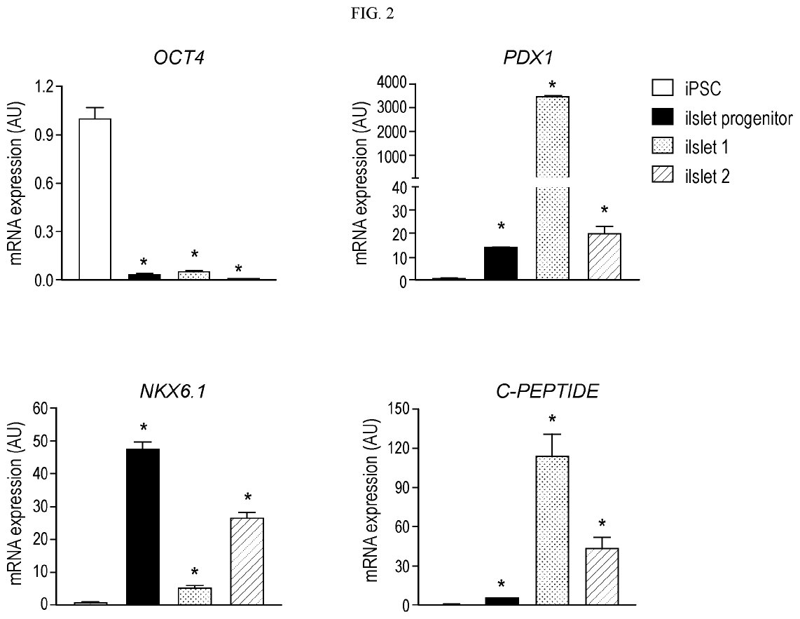

To investigate the contribution of specific cell populations of hiPSC-derived vascular endothelial cells (iECs) and pancreatic islets (iIslets) in enhancing generation of functional iIslets derived in vitro the Inventors have developed a robust and reproducible protocol to differentiate multiple lines of hiPSCs into islet (iIslet) progenitors in planar culture with high percentage of PDX1⁺/NKX6.1⁺ cells. For each cell line, at least 3 rounds of differentiation, and protein expression of 3 different cell lines were tested are shown in FIG. 1, demonstrating robustness of the method. Importantly, these cells presented increased mRNA expression of endocrine markers throughout their differentiation compared to iPSC stage, while decreased expression of pluripotency marker as shown in FIG. 2. iIslet progenitors with high expression of endocrine markers and some cells as with markers of β-cell progenitors (C-peptide) were generated, in addition to a high insulin content. However, these cells did not robustly secrete insulin upon repeated glucose challenges. Thus, the Inventors posited that by generating hiPSC-derived ECs (iECs) and co-culturing them with iIslet progenitors, the Inventors could attempt to improve functionality of the iIslets.

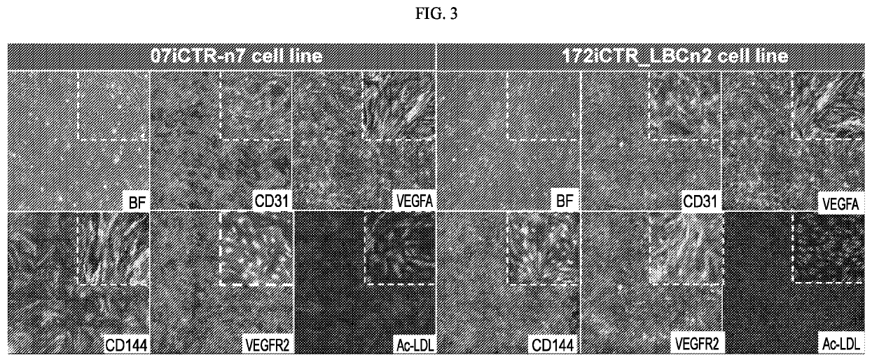

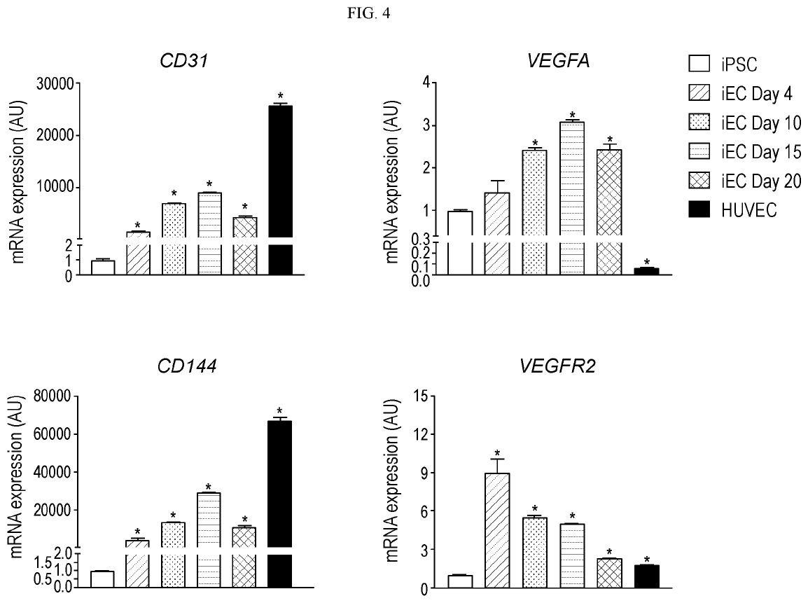

To generate iECs, the Inventors have developed a protocol, which resulted in iECs with high expression of multiple markers of the endothelium such as CD31, VEGF-A, VEGF-A receptor (VEGFR2) and CD144. The reproducibility and efficiency of this iEC protocol in multiple hiPSC lines (4 hiPSC lines) is shown day 20 from 2 cell lines (FIG. 3). In addition, the Inventors' iECs presented with increased mRNA expression of vascular EC markers throughout their differentiation compared to iPSC stage, which were similar to expression of markers of Human Umbilical Vein Cells (HUVECs), used as a positive control (FIG. 4). Importantly, these cells were functional as measured by the acetylated-low density lipoprotein (Ac-LDL) uptake from the media (FIG. 3), which is a functional assay that has been used to test the functionality of ECs for a long time.

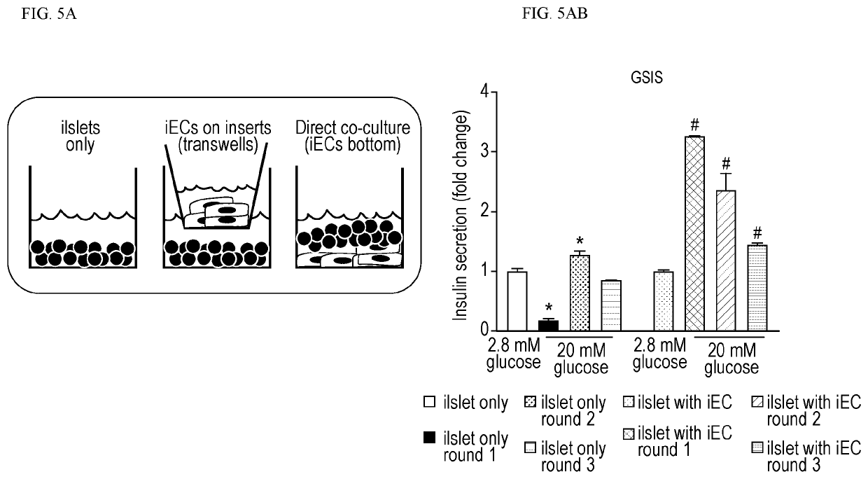

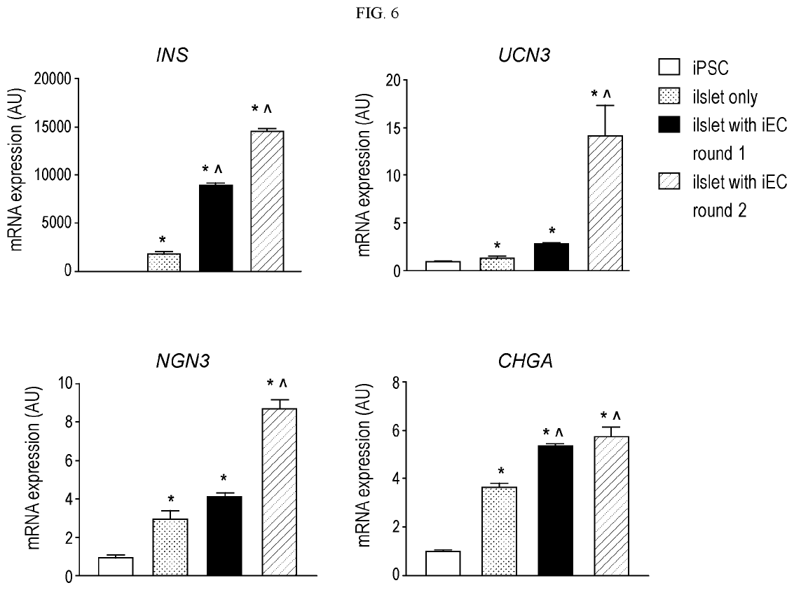

The Inventors then tested different methods of co-culturing iIslet progenitors with iECs (FIG. 5A). The Inventors' first attempt was to co-culture them using transwells, where signals are exchanged between cells through the media and there is no direct contact between cells. For this, iECs were introduced to same donor iIslet progenitors and they were co-cultured over 2 weeks, when iIslet progenitors were further developed to more mature iIslets. The Inventors tested iECs on the inserts and iIslet progenitors on the bottom of the plate and vice-versa. With this method, the Inventors observed higher expression of C-Peptide protein on iIslets at the end of the culture when iECs were cultured on the inserts and iIslets at the bottom of the plate. However, none of the methods of co-culturing iIslets with iECs on transwells improved iIslets functionality, since iIslets still had a great amount of insulin content, but they were not responsive to glucose challenges on a physiological fashion. Thus, based on prior evidence that cell-cell interaction between ECs and pancreatic cells is key during endocrine pancreas development, the Inventors applied direct co-culture of iIslet progenitors with same donor iECs again for 2 weeks. The Inventors tried different methods such as iECs on the bottom of the plate and iIslets on top of iECs and vice-versa. The Inventors found when iECs were on the bottom of iIslets for 2 weeks, iIslets at the end of the differentiation responded in a physiological fashion to glucose challenges, i.e. they increased insulin secretion when challenged to a high glucose concentration medium. This was not observed on iIslets when cultured alone (FIG. 5B). Importantly, iIslets co-cultured with iECs directly also presented with higher mRNA expression of multiple markers of mature pancreatic β-cells, such as insulin (INS), urocortin-3 (UCN3), neurogenin-3 (NGN3) and chromogranin-A (CHGA) when compared to iPSC stage and iIslets cultured alone (FIG. 6).

Collectively, these results show that co-culturing iIslet progenitors with iECs during islet development resulted in iIslets with improved maturity and functionality. However, the precise cell composition and signals exchanged between the iECs and iIslets contributing to enhanced iIslet function have not yet been investigated. Thus, the Inventors propose here to explore the nature of cell composition and mechanisms differently expressed in iIslets co-cultured with iECs through deep single cell transcriptomic analyses using single-cell RNA sequencing (sc RNA-seq). Single cell RNAseq has the ability to characterize rare endocrine and endothelial cell populations that are not captured by prior bulk analysis. With the results generated here, the Inventors will further interrogate the signaling pathways contributing to enhanced iIslet function by modulating significant pathways in vitro (gain and loss of function). Briefly, at the end of the co-culture, cells will be isolated into single cells and then follow the same steps as bulk RNAseq: reverse transcription, amplification, library generation and sequencing. Individual cells are encapsulated in individual droplets in a microfluidic device, where the reserve transcription reaction takes place. Each droplet carries a DNA barcode that uniquely labels the cDNA derived from a single cell. Once reverse transcription is complete, the cDNAs from many cells are mixed together for sequencing; the transcripts from a particular cell are identified by the unique barcode. PCA will be generated as well as IPA for pathway analyses.

The Inventors will perform single cell transcriptomic analyses using sc-RNAseq from the Inventors' samples in order to determine the cell composition and mechanisms differently expressed in iIslets co-cultured with iECs. Alternatively, the Inventors will interrogate the signaling pathways contributing to enhanced iIslet function by modulating significant pathways suggested by the Inventors' bulk mRNA-seq data and data on developmental pancreas in conjunction with the endothelium through gain and loss of function studies.

To Establish Novel Ex Vivo Dynamic Perfusion Systems to Test Functionality of Human iIslets

The Inventors found iIslets co-cultured with iECs presented an increased insulin secretion when challenged to a high concentration of glucose compared to iIslets cultured alone using a static assay (GSIS). To better characterize iIslets functionality in a more physiological fashion ex vivo, the Inventors will assess functionality of iIslets using dynamic GSIS in a novel 3D bioprinted vascularized iIslet platform, which consists of a perfusion system where iIslets are dynamically challenged with glucose through an iEC vascularized channel. This dynamic method is also useful for determining regulation of insulin release in response to various secretagogues, such as KCl, IBMX, tolbutamide, extendin-4 and l-arginine.

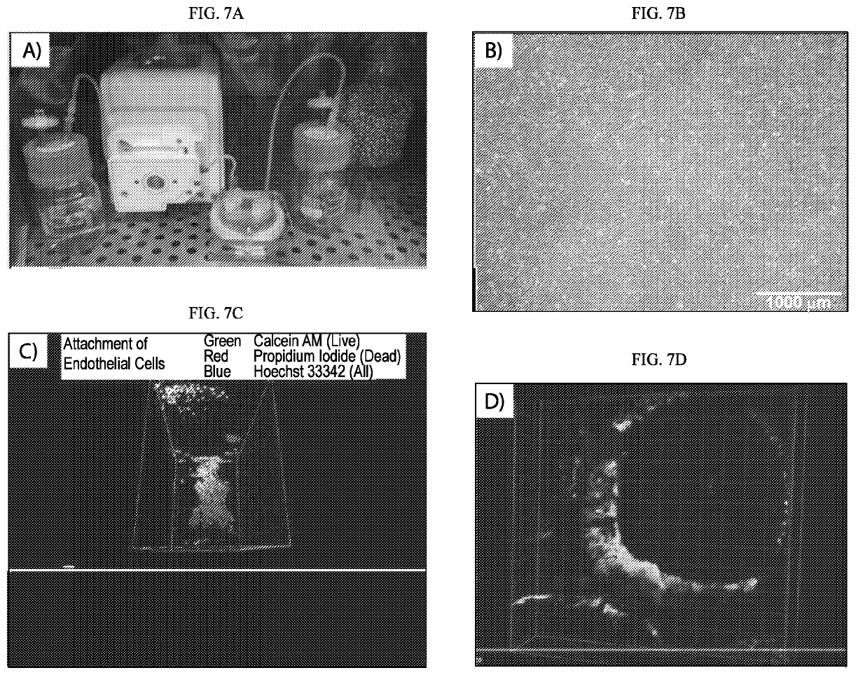

The Inventors have developed a 3D bioprinter with a motorized extruder that can precisely extrude and retract extrudate in a compact and rapidly loadable form-factor. In the past year, the Inventors have been culturing iECs on these constructs with success (FIG. 7). Briefly, iECs are cultured in planar from hiPSC until they reach maturity, when they are dissociated and mixed with bioinks (fibrin) to be bioprinted over the 3D constructs to resemble blood vessels. Cells are fed continuously through a dynamic system that mimics the blood flow on a living body (FIG. 7A), and the Inventors have observed that iECs spread out and remain attached to the constructs for more than 2 weeks. Here the Inventors propose a similar approach for iIslets co-cultured or not with iECs. At the end of their co-culture in planar, the Inventors will dissociate them, mix with appropriate bioink and bioprint them into 3D vessel constructs. Some studies have shown survival of islets in alginate bioinks and this will be the first approach to be tested. Other bioinks such as fibrin will also be tested as alternative methods. Viability of cells in the vascular constructs will be determined by Cell-Dead staining, where calcein acetoxymethylester (calcein AM) stains live cells while ethidiumhomodimer-2 stain labels non-viable cells, under a confocal microscope. iIslet functionality will then be tested through dynamic GSIS using this system after cells are attached and adapted to the system (the Inventors will test GSIS after different days from seeding on the constructs).

### Example 2

iPSC Generation

The iPSC lines utilized in this work were generated from healthy lean (BMI<27 kg/m²) male controls by the iPSC Core at Cedars-Sinai Medical Center. These control iPSC lines were generated from the peripheral blood mononuclear cells (PMBCs) utilizing non-integrating oriP/EBNA1-based episomal plasmid vectors. This approach resulted in <5% of abnormal karyotypes of iPSCs. All undifferentiated iPSCs were maintained in mTeSR⁺ media (StemCell Technologies) onto BD MATRIGEL™ matrix-coated plates. The cell lines used in this work are summarized at Table 1.

Differentiation of Pancreatic Islets from iPSCs (iIslets)

For the generation of pancreatic islets from iPSCs (iIslets), the Inventors have developed a robust and reproducible protocol. Briefly, iPSCs (OCT4 expression >90%) maintained on MATRIGEL-coated plates were single cell dissociated with Accutase and re-seeded with ROCK inhibitor Y-27632 (10 mM, R&D Systems) in planar onto MATRIGEL-coated plates at a density of 300,000 cells/cm² with mTeSR⁺ medium. 24-hr later, iPSCs were directed to definitive endoderm (DE—Phase I) using a combination of Activin-A (100 ng/ml, R&D Systems), CHIR99021 (2 μM, XcessBio) and Y-27632 (10 μM) for 1 day, followed by Activin-A (100 ng/ml) and FGF2 (5 ng/ml, PeproTech) for 2 days. Next, for the patterning of posterior foregut (PFG-Phase II), a combination of FGF10 (50 ng/ml, PeproTech), CHIR99021 (0.25 μM) and Noggin (50 ng/ml, PeproTech) was used for 2 days. To direct cells towards PDX1⁺/SOX9⁺ pancreatic progenitors (PP—Phase III), a combination of FGF10 (50 ng/ml), Noggin (50 ng/ml), RA (2 μM, Cayman) and SANT1 (0.25 μM, Sigma) was used for 4 days. Later, PDX1⁺/NKX6.1⁺ pancreatic endocrine progenitors (PEP—Phase IV) were induced through treatment with Noggin (50 ng/ml), EGF (100 ng/ml, PeproTech) and Nicotinamide (10 mM, Sigma) for 4 days. For the generation of islet progenitors (IP—Phase V), a combination of Noggin (50 ng/ml), T3 (1 μM, Sigma) and Alk5i II (10 μM, Axxora) was used for 7 days with media changes every other day. For the maturation of islets (MI—Phase VI), a combination of T3 (1 μM), Alk5i II (10 μM), AXL inhibitor R428 (2 Mm, Selleckchem) and antioxidant N-acetylcysteine NAC (1 mM, Sigma) was used for 14 days with media changes every other day. The formulation of the base media used throughout the differentiation is summarized at Table 2.

Differentiation of Vascular Endothelial Cells from iPSCs (iECs)

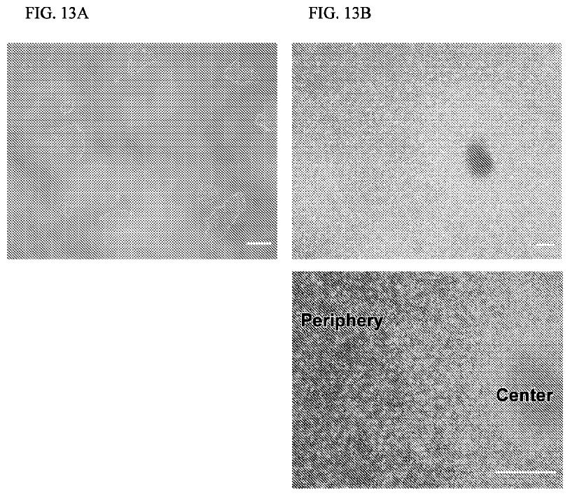

For the generation of vascular endothelial cells (ECs) from iPSCs (iECs), the Inventors plated iPSCs (OCT4 expression >90%) onto MATRIGEL-coated plates as small colonies of cells, and 3 days later (FIG. 13A) they were induced to mesoderm (ME—Phase I) using CHIR99021 (6 μM) for 2 days. Next, vascular progenitors (VP—Phase II) were generated using a combination of BMP4 (25 ng/ml, R&D Systems), FGF2 (10 ng/ml) and VEGF₁₆₅ (50 ng/ml, PeproTech) for another 2 days. After that, when the majority of the VPs presented a cobblestone-like morphology in the periphery of the original iPSC colonies (FIG. 13B), cells were dissociated with Accutase and the cells in the periphery easily lifted and were re-plated in planar onto MATRIGEL-coated plates at a density of 85,000-100,000 cells/cm² with VEGF₁₆₅ (50 ng/ml) and Y-27632 (10 μM) to induce EC progenitors (ECP—Phase III) for 7 days, changing media every other day. For purification and maturation of iECs (Phase IV), iECs were dissociated at Day 11 and re-plated at the same cell density onto MATRIGEL-coated plates with VEGF₁₆₅ (50 ng/ml) and media was changed every other day for 10 days. This process was repeated on Day 21 and extended for another 10 days if necessary. The base media used for Phases I and II was STEMdiff™ APEL™ 2 medium (StemCell Technologies) and for Phases III and IV, EC Growth medium MV2 (ECGM-MV2) (PromoCell).

Co-Culture Systems with iIslets and iECs

iIslets and iECs were co-cultures directly. For this, in parallel of Day 20 of iIslet differentiation, iECs Day 11 were plated on the bottom of 24-well MATRIGEL-coated plates using Phase IV EC media supplemented with Y-27632 (10 μM) until the next day. Day 21 iIslets were dissociated and plated on top of iECs at cell density of 400,000 cells/well using half Phase IV iEC media and half Phase VI islet media supplemented with Y-27632 (10 μM) (“iIslets with ½ iIslet media” condition), or they were fed with Phase VI media only (“iIslets with iIslet media” condition). The co-culture of iECs with iIslets was carried over 14 days, when cells were fixed for immunofluorescence or submitted to GSIS assay.

### Immunofluorescence

Cells were first fixed with 4% paraformaldehyde (PFA) in phosphate-buffered saline (PBS) for 20 minutes and subsequently washed 2× with PBS. Fixed cells were then permeabilized and blocked for 1 hour in a “blocking buffer” containing PBS with 10% donkey serum (Millipore) and 0.1% Triton-X (Bio-Rad). Primary antibodies were diluted in the blocking buffer and kept on cells overnight at 4° C. The following primary antibodies and dilutions were used: Oct4 (1:250, Stemgent), Ssea4 (1:100, Abcam), Foxa2 (1:100, Novus Biologicals), Sox17 (1:250, Novus Biologicals), Pdx1 (1:100, R&D Systems), Sox9 (1:250, Millipore), Nkx6.1 (1:25, DSHB), C-peptide (1:25, DSHB), Vegfa (1:100, Abcam), Cd31 (1:100, Cell Signaling), Cd144 (1:100, Abcam), Vegfr2 (1:100, Cell Signaling). The next day, after thorough washing using PBS with 0.1% Tween-20 (ThermoFisher), cells were incubated with appropriate species-specific Alexa Fluor-conjugated secondary antibodies (ThermoFisher) diluted in blocking buffer (1:1,000) for 1 hour at room temperature. After washing in PBS with 0.1% Tween-20, cells were incubated in DAPI diluted in PBS (1:2,500) for 15 min. Immunofluorescence images were visualized using appropriate fluorescent filters using ImageXpress Micro XLS (Molecular Devices) and analyzed using ImageJ Software.

### Static Glucose Stimulated Insulin Secretion (GSIS) Assay

The GSIS was performed at the end of iIslets differentiation and by the end of the co-culture of iIslets and iECs. For this, iIslets were washed 2× with 2.8 mM glucose Krebs solution (Table 4). Then, cells were pre-incubated with the same 2.8 mM glucose solution for 3 hours of fasting. After, cells were washed 1× with 2.8 mM glucose solution and incubated with the same solution for another 1 hour. After this incubation, the supernatant was collected as “low glucose”, and cells were washed 1× with 2.8 glucose solution, rinsed and challenged with a high glucose solution (20 mM glucose Krebs solution) (Table 4) for 1 hour. When this challenged was done, the supernatant was collected as “high glucose”. After this, cells were washed with PBS and fixed for immunofluorescence assay or collected for gene expression through RT-qPCR later on.

### Dil-Acetylated LDL-Uptake Assay

Dil fluorescent dye-labeled acetylated low density lipoprotein (Dil-ac-LDL) (10 μg/ml, Cell Applications) was added to Phase IV EC, HUVEC or mTeSR⁺ media and added to iEC Day 11 and 21, HUVECs or iPSCs accordingly, and incubated for 4 hours at 37° C., as described in (Harding et al., 2017). Cells were then washed with PBS, fixed and stained with DAPI, as described above. Images were taken with ImageXpress Micro XLS and analyzed using ImageJ.

### Real Time Quantitative PCR (RT-qPCR) Analysis

Relative gene expression was quantified using RT-qPCR. For this, cells were washed with PBS and the total RNA was extracted and isolated using Quick-RNA Mini Prep kit (Zymo Research), according to the manufacturer's instructions. The concentration and purity of RNA were determined by spectrophotometric analysis (NanoDrop, ThermoFisher), and all samples had a A_{260/280} ratio around 2.0 (Desjardins and Conklin, 2010). After, RNA (1 μg) was first DNAse treated (Promega and ThermoFisher), and then reverse transcribed to cDNA with oligo(dT) using the High Capacity cDNA Reverse Transcription kit (ThermoFisher). Real-time qPCR was performed in three replicates using SYBR Green Mastermix (Applied Biosystems) and specific primer sequences to each gene (Table 5), on a CFX384 Real Time system (Bio-Rad). Human RPL13 was used as reference gene and relative expression was determined using 2^{-ΔΔ} CT method.

### Statistical Analyses

Data are presented as mean±standard error of the mean (SEM). Statistical significance between groups was determined by One-way ANOVA followed by Dunnet post-test when compared to control, or Tukey's post-test for multiple comparisons test. Two-tailed unpaired Student's test was used as appropriate. P values <0.05 were considered statistically significant. Statistical analyses and graphs were generated using GraphPad Prism 7 for Windows Software (GraphPad Software).

Successful Generation of iPSC-Derived Pancreatic Endocrine Progenitors (PEPs)

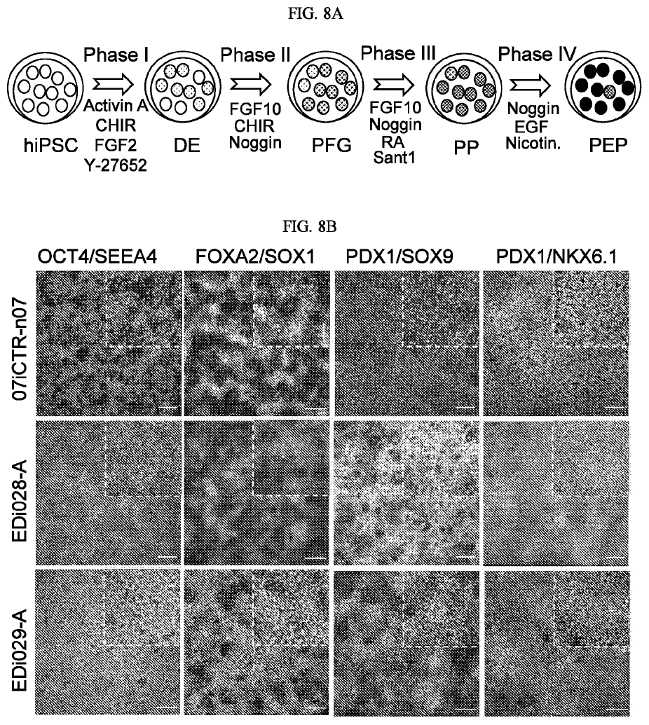

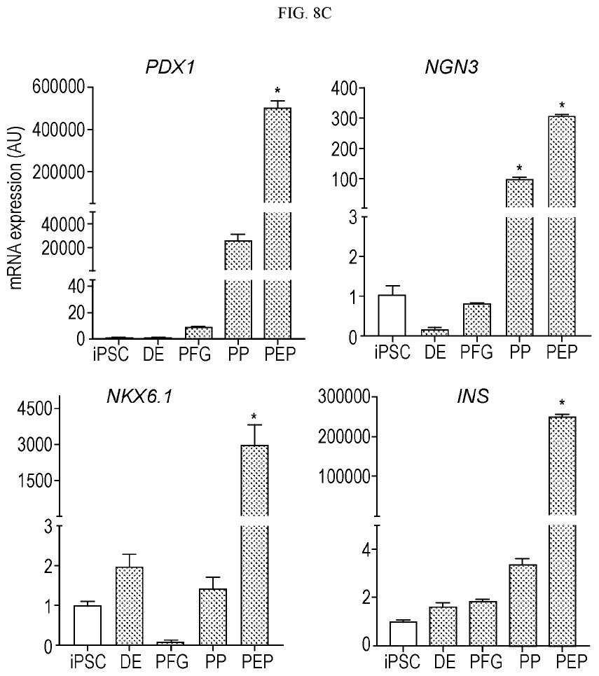

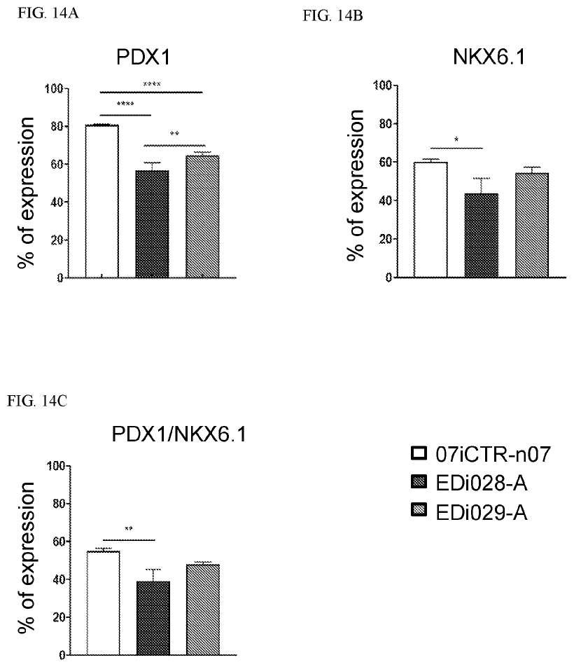

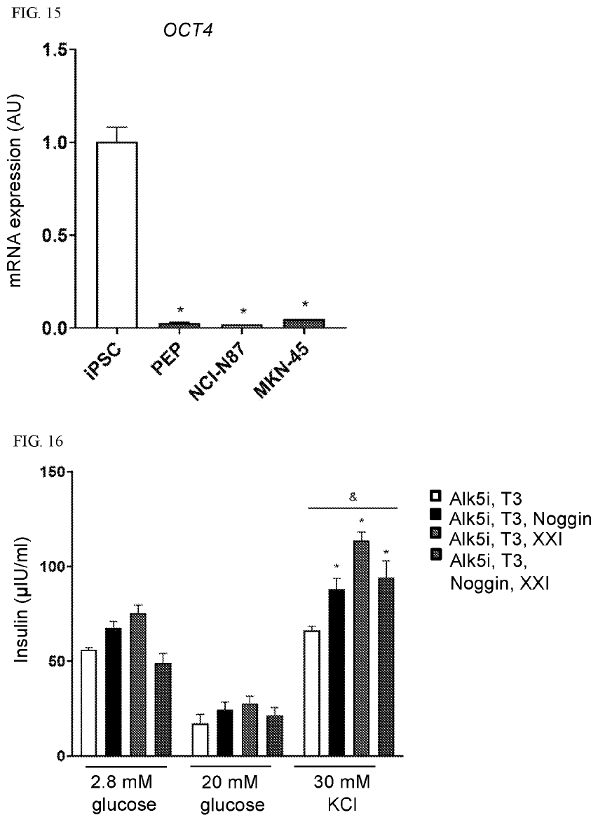

The differentiation strategies are summarized in FIG. 8A, where the small molecules used for each phase of differentiation are described in detail. After determining the optimal and most consistent cell density to initiate the differentiation, the Inventors demonstrated the Inventors' protocol to be consistent and reproducible in three different iPSC lines, as shown by protein expression of each phase of differentiation, as seen in FIG. 8B. Importantly, the Inventors have reached high protein expression of PDX1⁺/NKX6.1⁺ cells (>40%) at the end of Phase IV (FIG. 8B, FIG. 14), when cells were characterized as pancreatic endocrine progenitors (PEPs). The successful direction of cell fate into PEPs was also demonstrated by mRNA expression of further endocrine markers, i.e. NGN3 and INS, which was increased throughout the differentiation and reached their peak of expression at the end of Phase IV, as seen in FIG. 8C. As cells were being directed into PEPs, the expression of the pluripotent marker OCT4 concomitantly decreased, as shown in FIG. 15.

Inhibitory Combination of TGF-β and BMP Signaling Pathways Induced Generation of Islet Progenitors (IPs) from iPSC-Derived Pancreatic Endocrine Progenitors (PEPs)

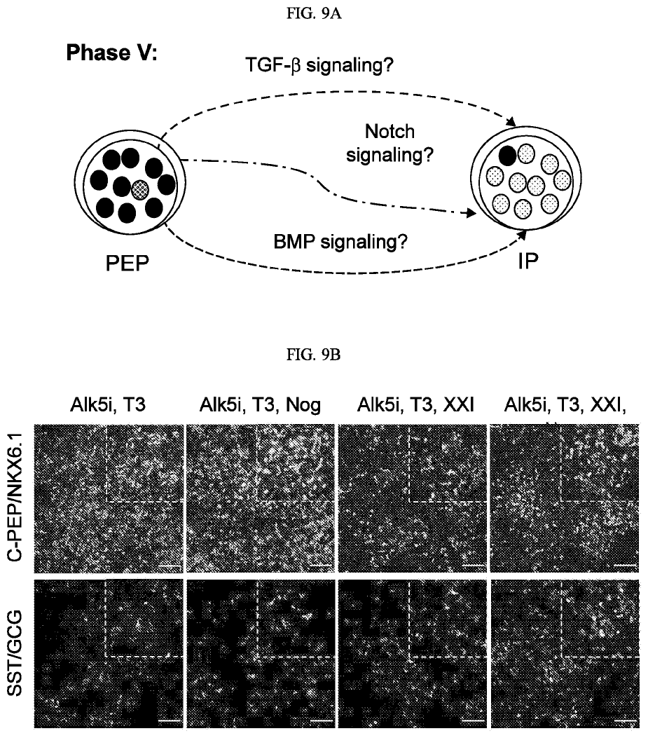

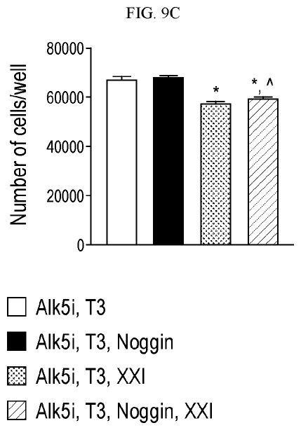

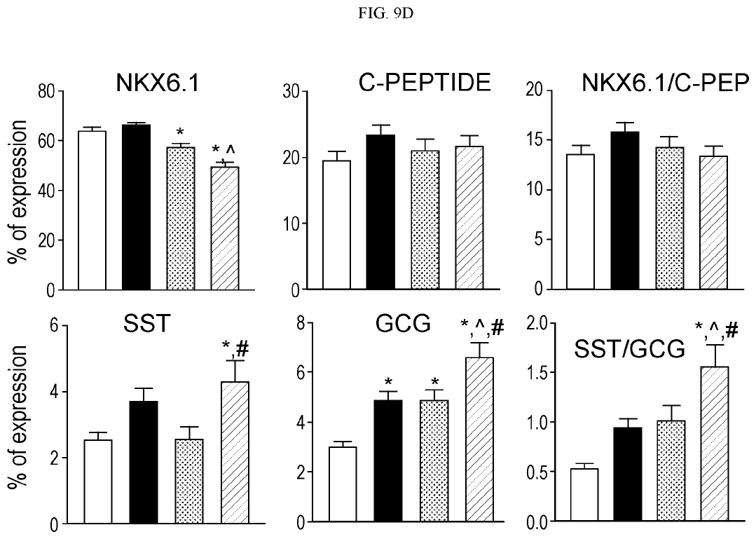

The Inventors' next step following the successful generation of PEPs across 3 different iPSC lines was to direct the cells into islet progenitors (IPs), more specifically β-cells, based on the Inventors' seminal observation that the genes NGN3 and INS, which are key regulators of pancreatic β-cell differentiation, were already highly expressed at the end of Phase IV of differentiation (FIG. 8C). This suggested to us that by the end of Phase IV, the Inventors' cells had already passed the pancreatic bipotent trunk progenitor phase, which can give rise to pancreatic endocrine or ductal cells, and reached the endocrine fate. Thus, based on previous literature, the Inventors interrogated three signaling pathways to direct PEPs into IPs: i) TGF-β inhibition, ii) BMP inhibition and/or iii) Notch inhibition, as described in FIG. 9A. The inhibition of TGF-β signaling is a strategy to achieve high expression of β-cells; here the Inventors tested the inhibition of TGF-β signaling using Alk5 II inhibitor (Alk5i). Another pathway that was modulated by us in order to direct PEPs into IPs was the inhibition of BMP signaling through the use of Noggin. It is well known that Noggin has a high affinity to BMPs, preventing their binding to their receptors. This blockage of BMP signaling results in either mitigation of β-cell proliferation or enhancement of β-cell proliferation and maintenance of β-cell specific gene expression. The Inventors' last strategy was to inhibit Notch signaling through the use of γ-secretase inhibitor XXI as an attempt to inhibit differentiation of remaining bipotent trunk progenitors into ductal cells. Across all conditions, T3 was used in an attempt to proliferate and mature β-cells, as indicated in previous studies.

In the different combinations tested during Phase V, Notch inhibition through the use of XXI resulted in a lower number of cells at the end of the Phase V compared to the conditions without XXI (FIGS. 9B and 9C), which could indicate less cell proliferation in the XXI conditions. Also, cells treated with XXI presented lower expression of NKX6.1, which is an important transcription factor present in pancreatic endocrine cells, and higher expression of markers of endocrine cell types other than β-cells, such as high expression of somatostatin (SST; δ-cells) and glucagon (GCG; α-cells). Among the conditions without XXI, the combination of T3, Alk5i and Noggin showed a tendency for higher C-PEPTIDE (P=0.05, t-Test), and NKX6.1/C-PEPTIDE (P=0.08, T-test) expression (FIGS. 9B and 9C).

To assess the functionality of β-cells, IPs at the end of Phase V were challenged to a static glucose stimulated insulin secretion (GSIS) assay, where cells receive different concentrations of glucose, and if functional, respond by secreting insulin in correlation with the glucose challenges. At the end of the GSIS, cells receive a solution containing a high concentration of KCl (30 mM KCl), which induces β-cell plasma membrane depolarization and can more potently stimulate insulin secretion than high concentrations of glucose. The results of the GSIS after Phase V can be seen in. FIG. 16, which shows no increase in insulin secretion after stimulation with a high concentration of glucose (20 mM glucose) in any conditions. However, after the challenge with 30 mM KCl, cells increased their insulin secretion in all conditions, indicating they were functional only after the KCl stimulation. In addition, the combination of T3, Alk5i and Noggin presented a higher insulin secretion when compared to T3 and Alk5i only (P=0.02, t-Test).

Because the GSIS results showed none of the conditions contained fully functional cells, and although the combination of T3, Alk5i and Noggin slightly presented higher expression of C-PEPTIDE and NKX6.1/C-PEPTIDE, and higher insulin secretion of KCl compared to the condition T3 and Alk5i only, the overall expression of C-PEPTIDE was not ideal (around 20-25% of cells), indicating a following phase after Phase V would be necessary to proliferate and mature IPs.

### Antioxidant N-Acetyl Cysteine and Inhibition of AXL Pathway Increased Maturation of Islet Progenitors (IPs), More Specifically β-Cells, but Did not Produce Functionality

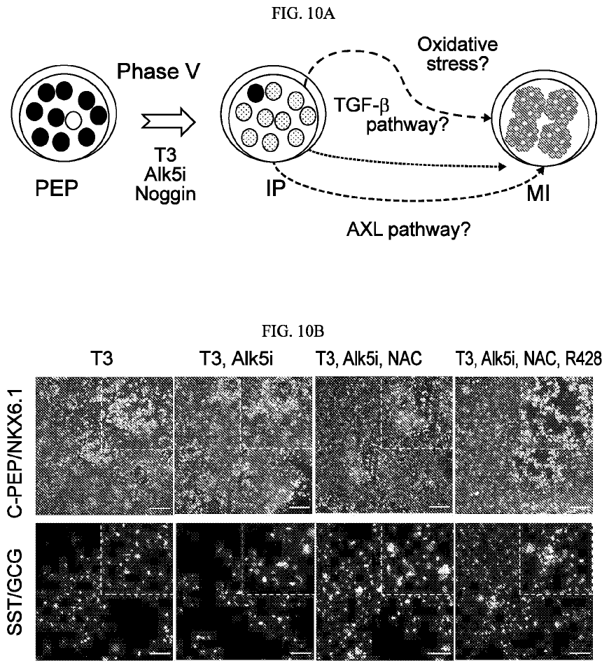

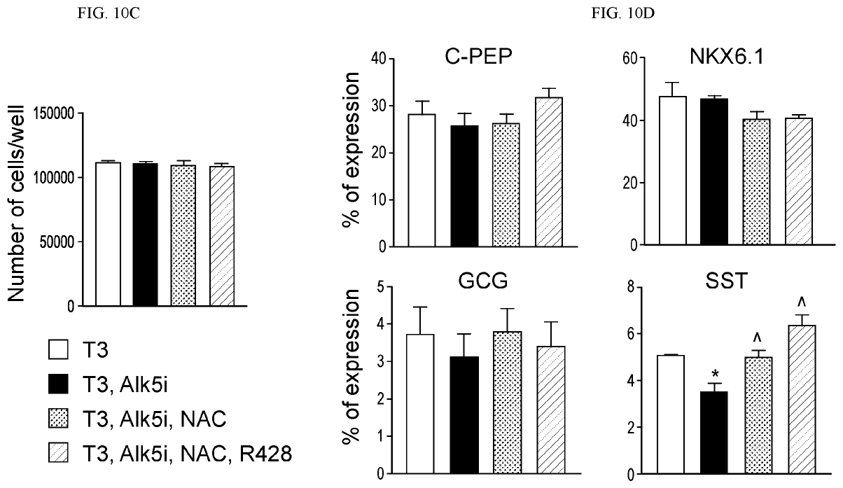

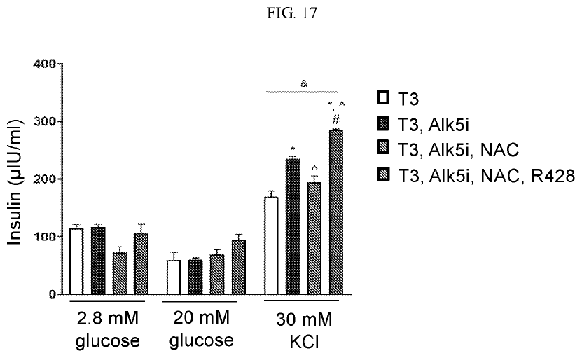

Although the combination of T3, Alk5i and Noggin used during Phase V directed PEPs into 20% C-PEPTIDE expressing populations and increased insulin secretion after stimulation with KCl, it was not enough to generate fully functional β-cells. Thus, as an attempt to mature IPs into islets containing functional β-cells, the Inventors modulated three main signaling pathways during Phase VI, based on previous literature: i) oxidative stress inhibition; ii) continuation of inhibition of TGF-β signaling; and iii) AXL pathway inhibition (FIG. 10A). The use of the selective small molecule inhibitor of the tyrosine kinase receptor AXL, R428, as well as the antioxidant N-acetyl cysteine (NAC) was based on the strategies used by (Rezania et al., 2014). These compounds have been identified as key regulators of genes that are important for β-cell maturation, including those involved in glucose-regulated insulin secretion (Rezania et al., 2014). After seven days of exposure to the Phase VI cocktail of small molecules, C-PEPTIDE protein expression increased compared to the end of Phase V, as observed in FIG. 10B-C, which shows C-PEPTIDE expression around 30%, and slightly decreased expression of somatostatin (SST) and glucagon (GCG), which are markers of other endocrine cells. The combination of T3, Alk5i, R428 and NAC showed higher expression of C-PEPTIDE (P=0.059, t-Test vs T3, Alk5i and NAC), although both conditions with R428 and NAC presented higher SST expression (FIG. 10B-C). Despite higher C-PEPTIDE expression compared to previous the Phase V, none of the conditions presented increased insulin secretion after stimulation with high glucose. However, when exposed to high a high concentration of KCl, all conditions increased insulin secretion, and the combination of T3, Alk5i, R428 and NAC presented the highest level of insulin secretion, as seen in FIG. 17. Thus, the combination of T3, Alk5i, R428 and NAC were used in further experiments.

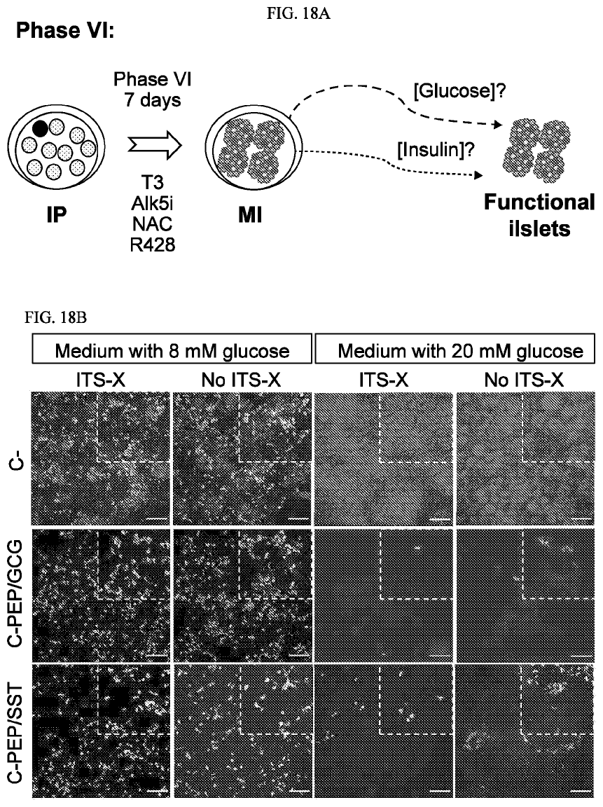

Due to the continued lack of functionality of β-cells despite higher C-PEPTIDE protein expression and higher insulin secretion after KCl stimulation, the Inventors then interrogated the concentration of insulin and glucose in the base medium of Phase VI as potential factors that could be mitigating β-cells maturation and response to glucose in a physiological fashion (FIG. 18A). For this, the Inventors probed the cells in Phase VI medium containing T3, Alk5i, R428 and NAC with 8 mM glucose (as before) or 20 mM glucose supplemented with insulin ITS-X (as before) or no glucose. When cells were treated with 20 mM glucose medium, the Inventors observed an absence of C-PEPTIDE expression after seven days of treatment and increased expression of NKX6.1 when compared to cells treated with 8 mM glucose medium, as seen in FIG. 18B, which could suggest that exposure of IPs to a medium with higher concentration of glucose for seven days reversed their differentiation direction towards a more progenitor state. In regard to the use of ITS-X or not, there was no difference in the expression of NKX6.1 or C-PEPTIDE proteins, as well as SST or GCG (FIG. 18B).

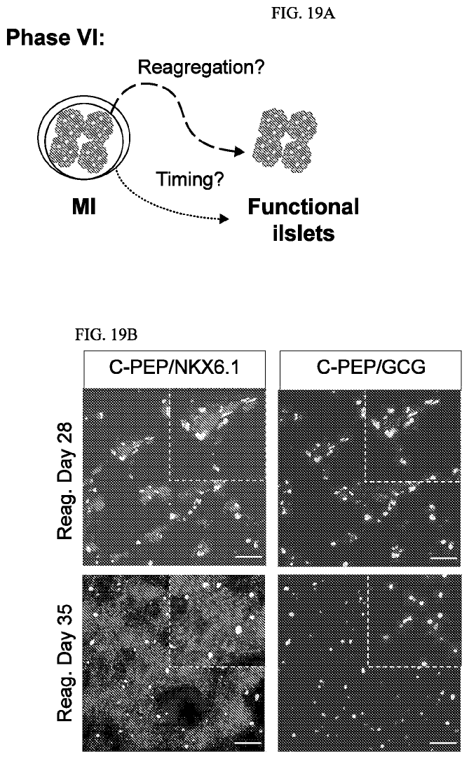

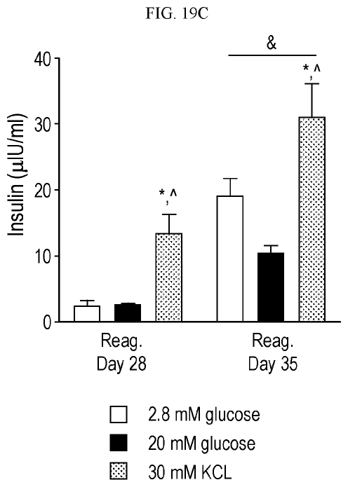

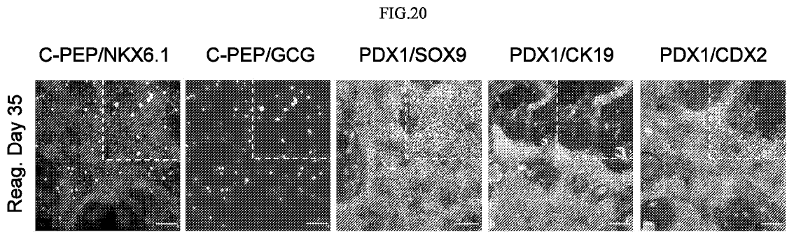

Since modulating the content of glucose and/or insulin in the medium at Phase VI was not enough to increase maturity of β-cells, the Inventors' next attempt to generate functional β-cells was to reaggregate cells during Phase VI and increase timing for differentiation (14 days instead of 7 days) (FIG. 97A). Dissociation and reaggregation of islet clusters is a strategy previously used by other studies as an attempt to purify the β-cell population within the islets and eliminate other cell types (Stock et al., 2020). Importantly, in these studies, reaggregation of β-cells improved GSIS (Lecomte et al., 2016). In the Inventors' work, cells were dissociated at Phase VI Day 1 and re-plated at the same initial cell density (3×10⁵ cells/cm²) in MATRIGEL-coated plates with Phase VI medium containing T3, Alk5i, R428 and NAC. The differentiation was carried out for 7 or 14 days. While C-PEPTIDE and GCG protein expression were similar between Day 28 (Phase VI day 7) vs Day 35 (Phase VI day 14), the expression of NKX6.1 was statistically higher at Day 35 (FIG. 19B). Although cells were still not fully functional when challenged to a high glucose solution, the baseline insulin levels after exposure of cells to 2.8 mM glucose was higher at Day 35 compared to Day 28 as well as the insulin response after the KCl challenge (FIG. 19C), which suggested to us that differentiation of β-cells carried out for 35 days was a better strategy to be taken in further steps. The development of other cell types, such as ductal and stomach/intestine cells, as shown in FIG. 20, might be linked to the absence of functionality at Day 35 after reaggregation.

Successful Generation of Functional iPSC-Derived Vascular Endothelial Cells (iECs)

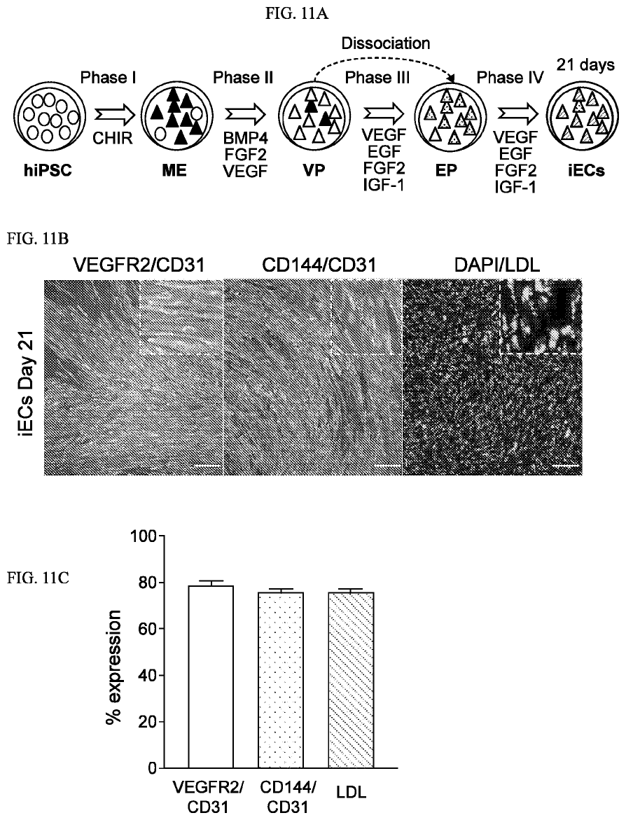

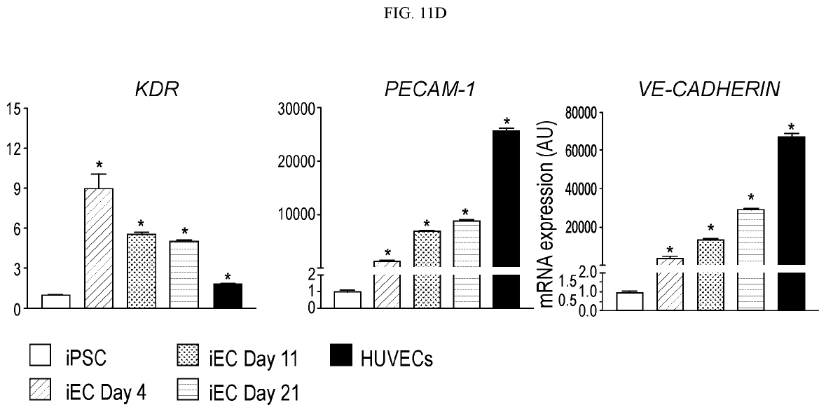

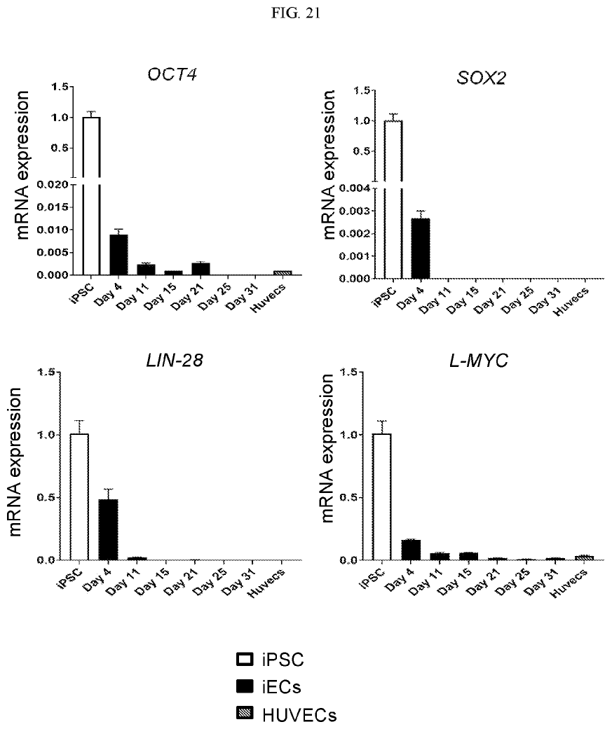

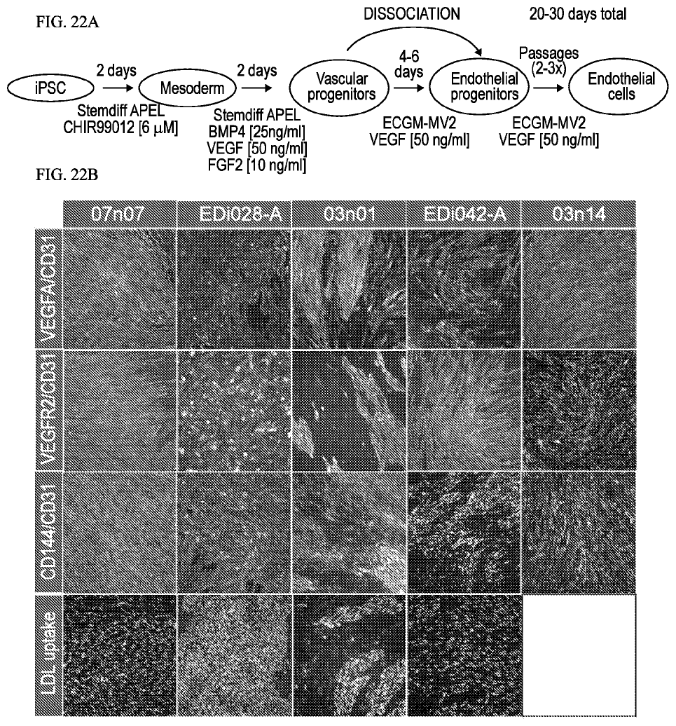

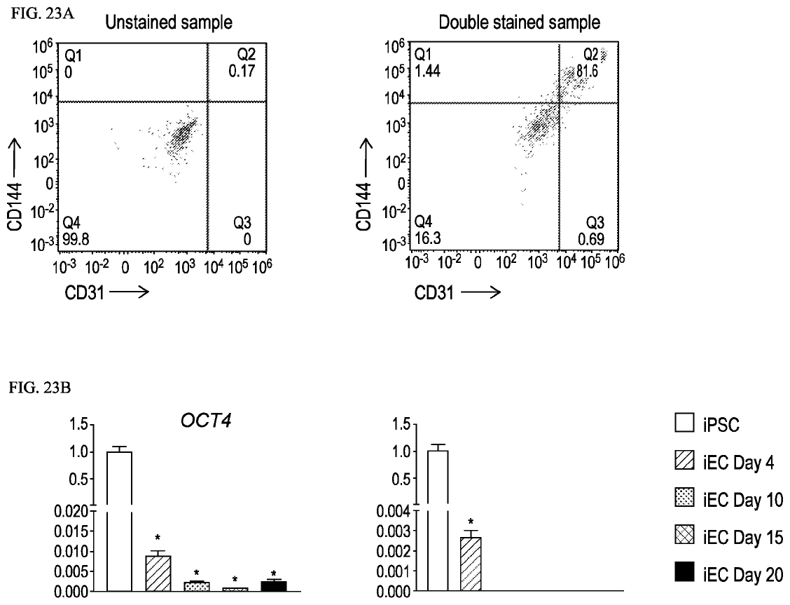

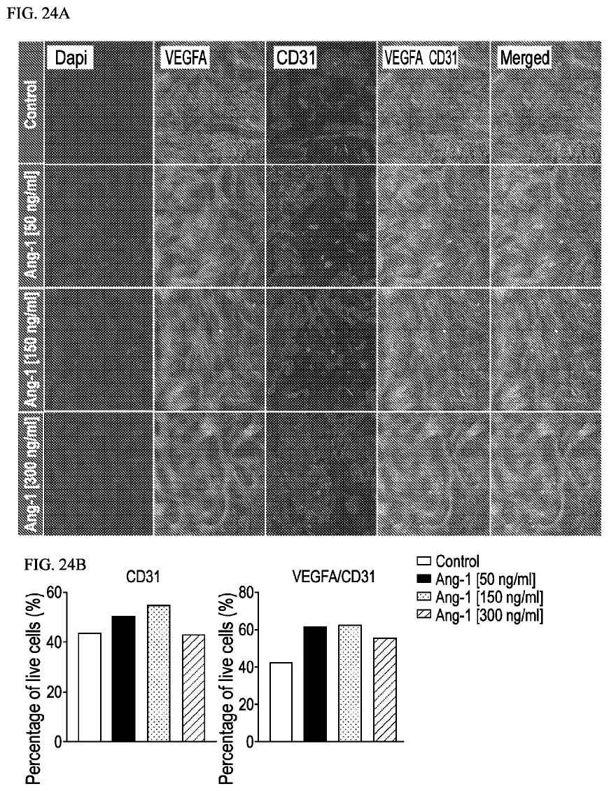

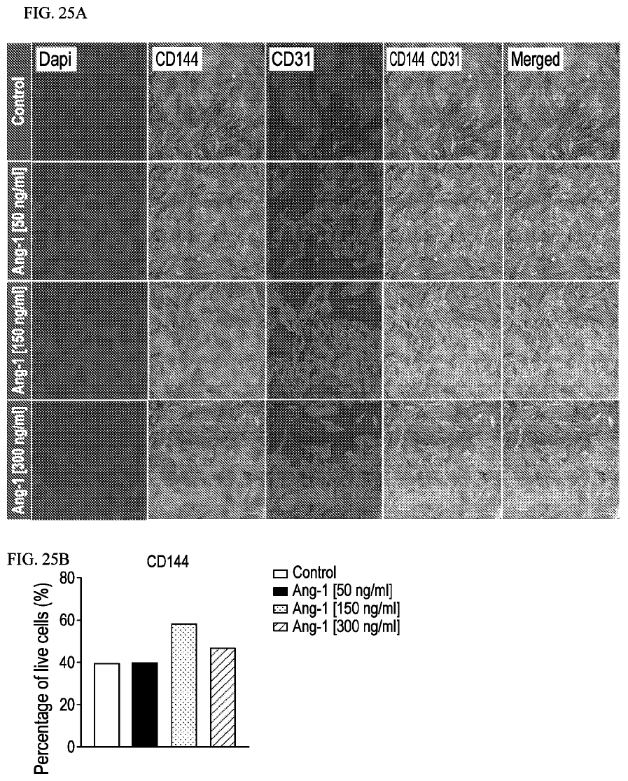

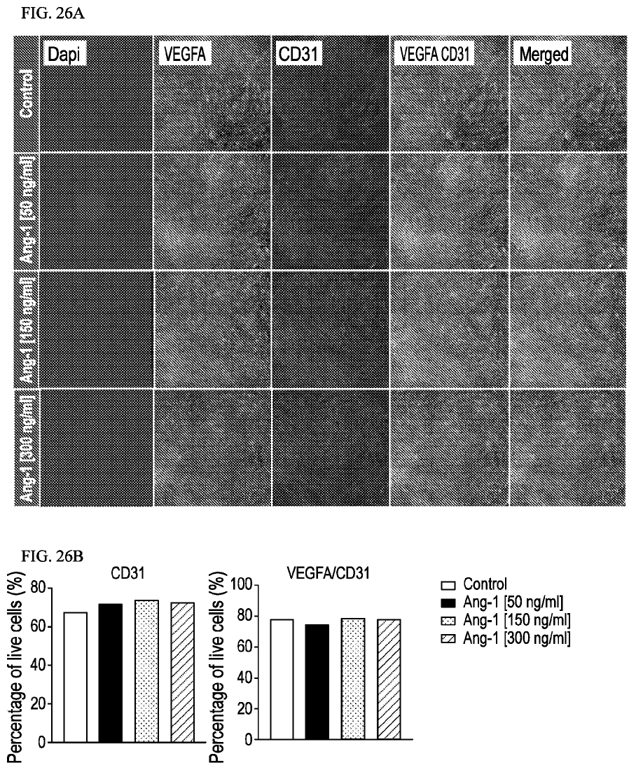

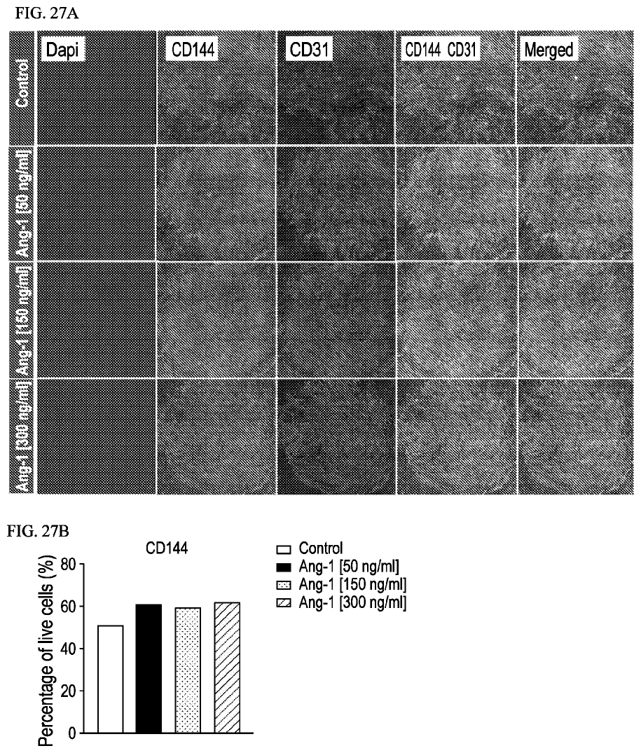

During endocrine pancreas development, surrounding vascular endothelial cells (ECs) provide signals for the development and maturation of pancreatic β-cells, which are considered a critical niche component. Due to the fact that the β-cells here differentiated presented high levels of C-PEPTIDE and NKX6.1 protein expression, but yet not optimal functionality, the Inventors believed that their co-culture with ECs could enhance their maturation and therefore, their functionality through exchanged signals. Since embodiments of the invention is to transplant iPSC-derived β-cells back to patients with diabetes, the Inventors used the same iPSC cell lines used for the β-cell differentiation to generate iPSC-derived ECs (iECs) for the co-culture. Thus, the Inventors have made several alterations to generate iECs, mainly related to alterations of cell density and plate coating (FIG. 11A). iPSC-derived vascular endothelial cells (iECs) generated by our optimized protocol presented high expression of endothelial cell markers such as VEGFR2, CD144 and CD31 (>70% by Day 21 of differentiation) (FIG. 11B, 11C). These cells were also functional as demonstrated by Dil-Ac-LDL uptake assay (FIG. 11B, 11C). They also highly expressed endothelial cell markers at the mRNA level, which increased throughout up to Day 21 (FIG. 11D). iECs presented minimal expression of pluripotency markers as shown in FIG. 21 from Day 4 to up to Day 31.

Optimal Model of Co-Culturing iPSC-Derived Islets (iIslets) with Endothelial Cells (iECs) Improved β-Cell Maturation and Functionality

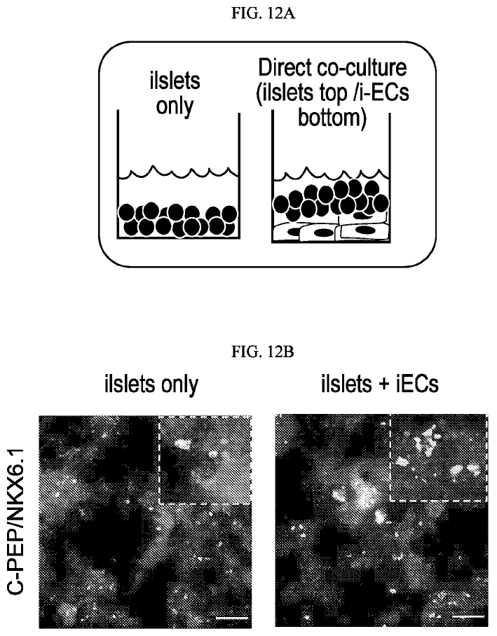

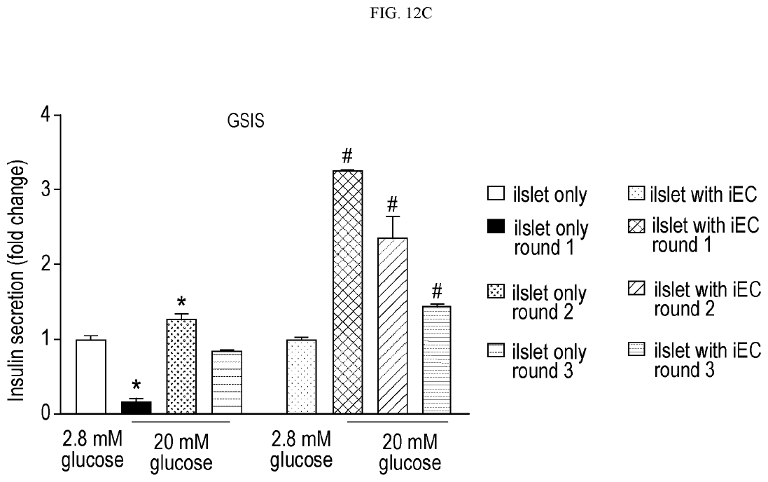

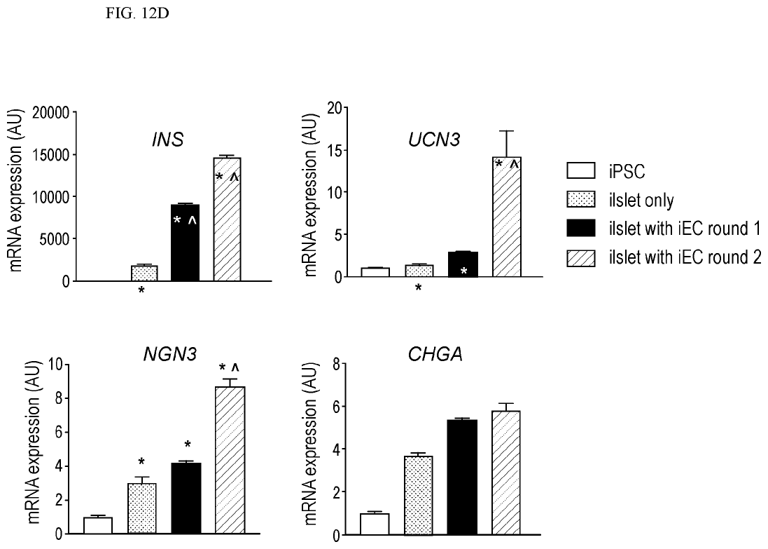

iPSCs from the same donor were differentiated up to Islet Progenitors (IP) as indicated in FIG. 2, using T3, Alk5i and Noggin. At the end of Phase V, IPs were dissociated using Accutase and re-plated on top of iECs Day 11 generated from the same iPSC line (07iCTR) at a cell density ratio of 1:4, as shown in the differentiation schematic of FIG. 12A. IPs were further differentiated into Mature Islets (MIs) as indicated in FIG. 3 using T3, Alk5i, R428 and NAC either in co-culture with iECs or not for 14 days. The combination of IPs and iECs was fed every other day with Phase VI media, as described previously. Alternatively, the co-culture of cells was fed with a combination of Phase VI media plus iEC media (50% each), but the results (not shown here) were not as promising as when the co-culture of cells was fed with Phase VI media only. The protein expression of C-PEPTIDE/NKX6.1 of “islets only condition” compared to “islets co-cultured with iECs” was not significantly different, as shown in FIG. 12B. However, when islets co-cultured with iECs were challenged to a high glucose concentration solution (20 mM glucose), they responded increasing their insulin secretion when compared to the basal glucose concentration solution (2.8 mM glucose) (FIG. 12C), while the “islets only condition” did not respond to the high glucose challenge, indicating the co-culture of islets with iECs activated mechanisms not yet elucidated that resulted in enhanced functionality of the islets. To confirm the enhanced functionality as probed by the GSIS shown in FIG. 12C, RNA was extracted from islets co-cultured either with iECs or not, and main markers expressed by islets containing mature β-cells were tested. Interestingly, there was an upregulation of the markers when islets were co-culture with iECs when compared to the “islets only condition”, such as INS, UCN3, NGN3 and CHGA (FIG. 12D). These results confirm the hypothesis based in earlier studies that the co-culture of pancreatic β-cells with ECs increases maturation and functionality of β-cells, and the mechanisms are yet to be elucidated.

TABLE 1
Characterization of the iPSC lines utilized in the work

| | | Parent | Disease | | | |
|---|---|---|---|---|---|---|
| Subject | Type | cell line | condition | Sex | Age | Race |
| CS0007iCTR-n07 | iPSC | PBMC | Control | Male | 60 | Unknown |
| CS0003iCTR-n01 | iPSC | PBMC | Control | Male | 34 | Asian |
| Edi028-A | iPSC | PBMC | Control | Male | 78 | White |
| EDi029-A | iPSC | PBMC | Control | Male | 79 | White |

* PBMC: Peripheral Blood Mononuclear Cell

TABLE 2
Formulation of base media for culture
of iPSC-derived pancreatic islets

| | | |
|---|---|---|
| | | Final |
| Component | Amount | concentration |

Phase I & II

| MCDB131 (FisherScientific) | 98 | ml | | |
|---|---|---|---|---|
| Glutamax (FisherScientific) | 1 | ml | 2 | mM |
| Vitamin C (Sigma) | 100 | μl | 250 | μM |
| BSA (VWR) | 500 | mg | 0.5% | |
| NaHCO₃ (Sigma) | 150 | mg | 1.5 | g/L |

| | | | |
|---|---|---|---|
| Pen/Strep (Sigma) | 1 | ml | 1% |

Phase III & IV

| DMEM (ThermoFisher) | 98 | ml | | |
|---|---|---|---|---|
| Vitamin C | 100 | μl | 250 | μM |

| | | | |
|---|---|---|---|
| B27 supplement without Vitamin A | 100 | μl | 1% |
| (ThermoFisher) | | | |
| Pen/Strep | 1 | ml | 1% |

Phase V

| MCDB131 | 98 | ml | | |
|---|---|---|---|---|
| Glucose (Sigma) | 360 | mg | 20 | mM |
| NaHCO₃ | 175.4 | mg | 1.754 | g/L |
| BSA | 2 | g | 2% | |
| ITS-X (ThermoFisher) | 500 | μl | 1:200 | |
| Glutamax | 1 | ml | 2 | mM |
| Heparin (Sigma) | 100 | μl | 10 | mg/L |
| Vitamin C | 100 | μl | 250 | μM |

| | | | |
|---|---|---|---|
| Pen/Strep | 1 | ml | 1% |

Phase VI

| MCDB131 | 98 | ml | | |
|---|---|---|---|---|
| Glucose | 44 | mg | 8 | mM |
| NaHCO₃ | 123 | mg | 1.23 | g/L |
| BSA | 2 | g | 2% | |
| ITS-X | 500 | μl | 1:200 | |
| Glutamax | 1 | ml | 2 | mM |
| Vitamin C | 100 | μl | 250 | μM |

| | | | |
|---|---|---|---|
| Pen/Strep | 1 | ml | 1% |

* BSA: Bovine Serum Albumin; ITS-X: Insulin-Transferrin-Selenium-Ethanolamine supplement.

TABLE 3
Formulation of media for culture of HUVECs culture

| Component | Amount | | Final concentration | |
|---|---|---|---|---|
| ATCC Vascular Cell Basal Medium | 98 | ml | | |
| Glutamax | 1 | ml | 2 | mM |
| Nonessential Amino acids (NEAAs) | 1 | ml | 1% | |
| (ThermoFisher) | | | | |
| Sodium Pyruvate (ThermoFisher) | 1 | ml | 1% | |
| Heparin | 100 | μl | 10 | mg/L |

| | | |
|---|---|---|
| Endothelial Cell Growth Supplement | 0.03-0.05 | mg/ml |
| (ECGS) (Corning) | | |

| | | | |
|---|---|---|---|
| FBS (ThermoFisher) | 20 | ml | 20% |
| Pen/Strep | 1 | ml | 1% |

* FBS: fetal bovine serum.

TABLE 4
Krebs buffer

| Component | Stock concentration [M] | Final concentration [mM] |
|---|---|---|
| NaCl | 5 | 128 |
| KCl | 2 | 5 |
| CaCl₂•2H₂O | 1 | 2.7 |
| MgSO₄ | 1 | 1.2 |
| Na₂HPO₄ | 0.1 | 1 |
| KH₂PO₄ | 1 | 1.2 |
| NaHCO₃ | 1 | 5 |
| Hepes | 1 | 10 |
| BSA | 25% | 0.1% |
| MilliQH₂0 | | |

* BSA: Bovine Serum Albumin

TABLE 5
List of oligonucleotides used for qPCR

| | | SEQ |
|---|---|---|
| Oligo | | ID |
| nucleotides | Primer sequence | NO |
| RPL13-F | GGCTAAACAGGTACTGCTGGG | 1 |
| RPL13-R | AGGAAAGCCAGGTACTTCAACTT | 2 |
| OCT4-F | GCAGAAGAGGATCACCCTGG | 3 |
| OCT4-R | TTGGCTGAATACCTTCCCAAA | 4 |
| PDX1-F | CCAGTGGGCAGGCGG | 5 |
| PDX1-R | AGGAACTCCTTCTCCAGCTCT | 6 |
| NKX6.1-F | GGCCTGTACCCCTCATCAAG | 7 |
| NKX6.1-R | TCCGGAAAAAGTGGGTCTCG | 8 |
| INS-F | AGGCCATCAAGCAGATCACT | 9 |
| INS-R | TTCCCCGCACACTAGGTAGA | 10 |
| NGN3-F | CCGGTAGAAAGGATGACGCC | 11 |
| NGN3-R | GGTCACTTCGTCTTCCGAGG | 12 |
| CHGA-F | ACTCCGAGGAGATGAACGGA | 13 |
| CHGA-R | TGGCTGCTCTGGTTCTCAAG | 14 |
| UCN3-F | CAGCCACAAGTTCATGGGGA | 15 |
| UCN3-R | ATCTCTCCCCGAGAGTGGAC | 16 |
| PCAM1-F | GGTCAGCAGCATCGTGGTCAACATAAC | 17 |
| PCAM1-R | TGGAGCAGGACAGGTTCAGTCTTTCA | 18 |
| VEGFR1-F | TCCCTTCCTTCAGTCATGTGT | 19 |
| VEGFR1-R | AAGAAGGAAACAGAATCTGCAA | 20 |
| VEGFR2-F | AGCCATGTGGTCTCTCTGGTTGTGTATG | 21 |
| VEGFR2-R | GTTTGAGTGGTGCCGTACTGGTAGGA | 22 |
| VE-Cadherin-F | CATCTTCCCAGGAGGAACAG | 23 |
| VE-Cadherin-R | AGAGCTCCACTCACGCTCAG | 24 |
| VEGFA-F | CTCCACCATGCCAAGTGGTC | 25 |
| VEGFA-R | GCAGTAGCTGCGCTGATAGA | 26 |

TABLE 6
Formulation of media for culture of iPSC-derived endothelial cells

| | | | Final | | Phase of |
|---|---|---|---|---|---|
| | | | concen- | | differen- |
| Day | Base medium | Component | tration | | tiation |
| −3 to −1 | mTeSR+ | — | — | | iPSC |
| 0 to 1 | STEMdiff ™ | CHIR99021 | 6 | μM | Mesoderm |
| | APEL ™2 | | | | |
| 2 to 3 | STEMdiff ™ | BMP4 | 25 | ng/ml | Vascular |
| | APEL ™2 | FGF2 | 10 | ng/ml | progenitor |
| | | VEGF₁₆₅ | 50 | ng/ml | |
| 4 to 11 | ECGM MV2 | VEGF₁₆₅ | 50 | ng/ml | Endothelial |
| (change | | | | | progenitor |
| media every | | | | | |
| other day) | | | | | |
| 11 to 21 | ECGM MV2 | VEGF₁₆₅ | 50 | ng/ml | Mature |
| (change | | | | | endothelial |
| media every | | | | | cell |
| other day) | | | | | |

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without are the compositions and methods related to iPSC, islet cells, endothelial cells, and, techniques and composition and use of solutions used therein, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method of generating functional induced pluripotent stem cell (iPSC) derived pancreatic islets (iIslets), comprising:
co-culturing a quantity of iPSC derived vascular endothelial cells (iECs) and a quantity of iPSC derived islet progenitors for about 10-18 days to generate the functional iIslets comprising β-cells.

2. The method of claim 1, wherein co-culturing comprises:
plating a quantity of iPSC derived vascular endothelial cells (iECs) on MATRIGEL-coated plates and culturing in Phase IV EC media supplemented with Y27632;
plating a quantity of iPSC derived pancreatic islets (iIslets) on top of the quantity of iECs and either culturing in media comprising about ½ Phase IV iEC media and about ½ Phase VI islet media supplemented with Y-27632 for about 12-16 days, or culturing in Phase VI islet media (islet only condition) for about 12-16 days, to generate the functional iIslets comprising β-cells.

3. (canceled)

4. The method of claim 1, further comprising generating the iECs by:
plating a quantity of induced pluripotent stem cells (iPSCs) onto MATRIGEL;
culturing the iPSC in MATRIGEL for about 2-4 days;
culturing in the presence of CHIR99021 for about 1-3 days to generate mesoderm;
culturing the mesoderm in the presence of BMP4, FGF2, and VEGF for about 1-3 days to generate vascular progenitors;
culturing the vascular progenitors in the presence of VEGF and Y-27632 for about 3-8 days to generate endothelial cell (EC) progenitors.

5. (canceled)

6. The method of claim 1, further comprising first generating the quantity of islet progenitors by:
culturing a quantity of induced pluripotent stem cells (iPSCs) in the presence of Activin-A, CHIR99021 and Y-27632 for about 1-2 days;
culturing in the presence of Activin-A and FGF2 for about 1-3 days;
culturing in the presence of FGF10, CHIR99021 and Noggin for about 1-3 days, to generate posterior foregut cells;
culturing the posterior foregut cells in the presence of FGF10, Noggin, RA and SANT1 for about 3-5 days to generate pancreatic progenitors;
culturing the pancreatic progenitors in the presence of Noggin, EGF and Nicotinamide for about 3-5 days to generate pancreatic endocrine progenitors;
culturing the pancreatic endocrine progenitors in the presence of Noggin, T3 and Alk5i II for about 6-8 days to generate islet progenitors.

7. (canceled)

8. The method of claim 1,
wherein the pancreatic progenitors express PDX1+ and SOX9+, or
wherein the pancreatic endocrine progenitors are PDX1+ and NKX6.1+, or
wherein the iIslets express C-peptide, glucagon and NKX6.1+.

9. (canceled)

10. (canceled)

11. The method of claim 2, wherein, the expression of INS, UCN3, NGN3 and CHGA are upregulated in the β-cell that are produced in the islets only condition, as compared to β-cell that are produced without co-culturing with vascular endothelial cells or as compared to β-cell that were produced in a culture without the islets only condition.

12. The method of claim 1,
wherein the β-cell increase insulin secretion when challenged with a high glucose concentration as compared to a basal glucose concentration, or
wherein the iPSC derived vascular endothelial cells (iECs) and iPSC derived islet progenitors are isogenic, or
wherein the iPSCs used to derive vascular endothelial cells (iECs) and iPSC used to derive islet progenitors are from the same iPSC cell line or from the same donor, or
wherein the iIslets are human iIslets.

13. (canceled)

14. (canceled)

15. (canceled)

16. Induced pluripotent stem cell (iPSC) derived pancreatic islets (iIslets) produced by a method of claim 1.

17. Induced pluripotent stem cell (iPSC) derived pancreatic islets (iIslets) expressing C-peptide, glucagon and NKX6.1+.

18. The iIslets of claim 16, wherein the iIslets increase insulin secretion when challenged with a high glucose concentration as compared to a basal glucose concentration.

19. A method of ameliorating or treating a metabolic disease, metabolic disorder or metabolic condition in a subject in need thereof, comprising:
administering iIslets of claim 16 to the subject in need thereof to ameliorate or treat the metabolic disease, metabolic disorder or metabolic condition.

20. The method of claim 19, wherein the metabolic disease, metabolic disorder or metabolic condition is diabetes or insulin resistance.

21. A method, comprising:
culturing a quantity of induced pluripotent stem cells (iPSCs) in the presence of Activin-A, CHIR99021 and Y-27632 for about 1 day;
followed by culturing in the presence of Activin-A and FGF2 for about 2 days; and
followed by culturing in the presence of FGF10, CHIR99021 and Noggin for about 2 days, to generate posterior foregut cells.

22. The method of claim 21, further comprising culturing the posterior foregut cells in the presence of FGF10, Noggin, RA and SANT1 for about 4 days to generate pancreatic progenitors.

23. The method of claim 22, wherein the pancreatic progenitors express PDX1⁺ and SOX9⁺.

24. The method of claim 21, further comprising culturing the pancreatic progenitors in the presence of Noggin, EGF and Nicotinamide for about 4 days to generate pancreatic endocrine progenitors.

25. The method of claim 21, wherein the pancreatic endocrine progenitors are PDX1⁺ and NKX6.1⁺.

26. The method of claim 24, further comprising culturing the pancreatic endocrine progenitors in the presence of Noggin, T3 and Alk5i II for about 7 days to generate islet progenitors.

27. The method of claim 26, further comprising culturing the generated islet progenitors in the presence of T3, Alk5i II, R428, and N-acetylcysteine (NAC) for about 14 days to generate mature islets.

28. The method of claim 27, wherein the mature islets express C-peptide, glucagon and NKX6.1⁺.

29. The method of claim 21, further comprising:
culturing the posterior foregut in the presence of FGF10, Noggin, RA and SANT1 for about 4 days to generate pancreatic progenitors;
followed by culturing the pancreatic progenitors in the presence of Noggin, EGF and Nicotinamide for about 4 days to generate pancreatic endocrine progenitors;
followed by culturing the pancreatic endocrine progenitors in the presence of Noggin, T3 and Alk5i II for about 7 days to generate islet progenitors; and
followed by culturing the generated islet progenitors in the presence of T3, Alk5i II, R428, and N-acetylcysteine (NAC) for about 14 days to generate mature islets.

30. A quantity of mature islets made by the method of claim 27.

31. A method, comprising:
plating a quantity of induced pluripotent stem cells (iPSCs) onto MATRIGEL;
culturing for about iPSC in MATRIGEL for about 3 days; and
followed by culturing in the presence of CHIR99021 to generate mesoderm.

32. The method of claim 31, further comprising culturing the mesoderm in the presence of BMP4, FGF2, and VEGF for about 2 days to generate vascular progenitors.

33. The method of claim 32, further comprising culturing the vascular progenitors in the presence of VEGF and Y-27632 for about 7 days to generate endothelial cell (EC) progenitors.

34. The method of claim 33, further comprising culturing the EC progenitors with VEGF for about 10 days to generate mature EC.

35. The method of claim 34, wherein the mature EC express CD31⁺, CD144⁺, VEGF-A⁺, VEGFR2⁺, and Ac-LDL.

36. The method of claim 31, further comprising:
culturing the mesoderm in the presence of BMP4, FGF2, and VEGF for about 2 days to generate vascular progenitors;
followed by culturing the vascular progenitors in the presence of VEGF and Y-27632 for about 7 days to generate endothelial cell (EC) progenitors; and
followed by culturing the EC progenitors with VEGF for about 10 days to generate mature EC.

37. A quantity of mature EC made by the method of claim 34.

38. An assembly, comprising a quantity of mature islets and a quantity of mature EC, wherein the mature islets and the mature EC are isogenic.

39. The assembly of claim 38,
wherein the quantity of the mature islets made by a process comprising co-culturing a quantity of iPSC derived vascular endothelial cells (iECs) and a quantity of iPSC derived islet progenitors for about 10-18 days to generate mature iIslets comprising β-cells, and
wherein the quantity of the mature ECs is made by a process comprising:
plating a quantity of induced pluripotent stem cells (iPSCs) onto MATRIGEL;
culturing for about iPSC in MATRIGEL for about 3 days;
followed by culturing in the presence of CHIR99021 to generate mesoderm;
culturing the mesoderm in the presence of BMP4, FGF2, and VEGF for about 2 days to generate vascular progenitors;
culturing the vascular progenitors in the presence of VEGF and Y-27632 for about 7 days to generate endothelial cell (EC) progenitors; and
culturing the EC progenitors with VEGF for about 10 days to generate mature EC.

40. The assembly of claim 38, wherein the mature islets, the mature EC, or both, are deposited on a scaffold.

41. The assembly of claim 40, wherein the mature islets, mature EC or both, are deposited on the scaffold using a bioink.

42. The assembly of claim 41, wherein the bioink comprises fibrin or alginate.
